SEQUENCE LISTING (14 sequences)

SEQ ID NO: 1 - DNA, 46 bp, Artificial Sequence
  Synthetic Construct
aaccactttgtacaagaaagcttagttttcagaagcagtagctctg

SEQ ID NO: 2 - DNA, 46 bp, Artificial Sequence
  Synthetic Construct
cagagctactgcttctgaaaactaagctttcttgtacaaagtggtt

SEQ ID NO: 3 - DNA, 52 bp, Artificial Sequence
  Synthetic Construct
cagattaggtagtcgaaaaacctaagaaatttaggtgctacatttcaagatt

SEQ ID NO: 4 - DNA, 52 bp, Artificial Sequence
  Synthetic Construct
aatcttgaaatgtagcacctaaatttcttaggtttttcgactacctaatctg

SEQ ID NO: 5 - DNA, 21 bp, Artificial Sequence
  Synthetic Construct
ggacuuaucaaacugguuutt

SEQ ID NO: 6 - DNA, 21 bp, Artificial Sequence
  Synthetic Construct
aaaccaguuugauaagucctt

SEQ ID NO: 7 - DNA, 18 bp, Artificial Sequence
  Synthetic Construct
atgccgcctaaggacgac

SEQ ID NO: 8 - DNA, 18 bp, Artificial Sequence
  Synthetic Construct
tcatgcatcttcaccagc

SEQ ID NO: 9 - DNA, 18 bp, Artificial Sequence
  Synthetic Construct
gcactcttccagccttcc

SEQ ID NO: 10 - DNA, 19 bp, Artificial Sequence
  Synthetic Construct
gcgctcaggagggagcaat

SEQ ID NO: 11 - RNA, 19 nt, Homo sapiens
ggacuuaucaaacugguuu

SEQ ID NO: 12 - DNA, 67 bp, Artificial Sequence
  Synthetic Construct
cgcgtccccggacttatcaaactggtttttcaagagaaaaccagtttgataagtcctttttggaaat

SEQ ID NO: 13 - DNA, 65 bp, Artificial Sequence
  Synthetic Construct
cgatttccaaaaacctgaatagtttgaccaaaagagaactttttggtcaaactattcaggcccct

SEQ ID NO: 14 - RNA, 7 nt, Artificial Sequence
  Synthetic Construct
uagccau

(19) United States
(12) Patent Application Publication
(10) Pub. No.: US 2012/0065247 A1
(43) Pub. Date: Mar. 15, 2012

(54) MODULATING IRES-MEDIATED TRANSLATION

(75) Inventors: Sunnie R. Thompson, Birmingham, AL (US); Erik Mills Schwiebert, Birmingham, AL (US); John H. Streiff, Birmingham, AL (US)

(73) Assignees: DiscoveryBiomed, Inc., Birmingham, AL (US); The UAB Research Foundation, Birmingham, AL (US)

(21) Appl. No.: 13/259,503

(22) PCT Filed: Mar. 26, 2010
(86) PCT No.: PCT/US2010/028917

§ 371 Date: Oct. 28, 2011

Related U.S. Application Data
(60) Provisional application No. 61/164,167, filed on Mar. 27, 2009.

(51) Int. Cl. A61K 31/7088; C07C 235/56; C07D 277/46; C07D 213/46; C07C 217/60; C07D 231/12; A61K 31/44; A61K 31/4406; A61K 31/426; A61K 31/415; A61K 31/167; A61K 31/137; A61K 31/713; A61P 31/14; A61P 31/22; A61P 31/20; C12N 5/071; A61P 35/00; C12Q 1/02; C12Q 1/68; C12Q 1/70; C12Q 1/66; A61K 31/4402; C07D 213/75

(52) U.S. Cl. 514/44 A; 546/309; 564/176; 548/195; 546/338; 546/285; 564/336; 548/377.1; 514/352; 514/357; 514/371; 514/406; 514/622; 514/649; 435/375; 435/29; 435/6.13; 435/5; 435/6.11; 435/8

(57) ABSTRACT

Provided herein are compounds and methods for use in preventing or treating a viral infection mediated by a virus comprising an IRES-containing RNA molecule or cancer related to an increase or decrease in IRES-mediated translation of an RNA molecule. Also provided are methods of inhibiting or promoting IRES-mediated translation. Also provided are methods of screening for an agent that inhibits IRES-mediated translation.

### CROSS REFERENCE TO RELATED APPLICATIONS

This application claims priority to U.S. Provisional Application No. 61/164,167, filed Mar. 27, 2009, which is incorporated herein in its entirety.

### STATEMENT REGARDING FEDERALLY FUNDED RESEARCH

The present invention was made with support from Grant Nos. CM084547 and 5T32HL007553 from the National Institutes of Health and Grant No. CA-13148-31 from the National Cancer Institute. The U.S. Government has certain rights in this invention.

### BACKGROUND

The vast majority of messenger RNAs (mRNAs) are translated using a cap-dependent mechanism of translation. However, 5-10% of messages initiate translation using a cap-independent mechanism that is not as well defined. mRNAs that contain an internal ribosome entry site (IRES) located in the 5′ untranslated region are able to initiate translation by a cap-independent mechanism.

### SUMMARY

Provided are compounds and methods for use in preventing or treating a viral infection mediated by a virus comprising an internal ribosome entry site (IRES)-containing RNA molecule or a cancer related to increased or decreased IRES-mediated translation of a mRNA molecule. The methods comprise identifying a subject with or at risk of developing a viral infection mediated by a virus comprising an internal ribosome entry site (IRES)-containing RNA molecule and administering to the subject a therapeutically effective amount of any of the compounds provided herein. The compound may or may not reduce ribosome protein S25 (Rps25) expression or function. Thus, the methods comprise or further comprises administering to the subject a therapeutically effective amount of an agent that reduces Rps25 expression or function.

Also provided are methods of inhibiting IRES-mediated translation. Specifically provided is a method comprising providing a cell, wherein the cell comprises an IRES-comprising RNA molecule, and contacting the cell with an agent, wherein the agent reduces ribosomal protein S25 (Rps25) expression or function in comparison to a control. The method can further comprise determining that IRES-mediated translation is inhibited by detecting a reduced level of protein expressed by the IRES-containing RNA molecule in comparison to a control.

Also provided are methods of screening for an agent that inhibits IRES-mediated translation. Specifically provided is a method comprising providing a system that includes a Rps25 or a nucleic acid that encodes Rps25 and an IRES-containing RNA molecule, contacting the system with the agent to be tested, and determining Rps25 expression or function. A decrease in the level of Rps25 expression or function indicates the agent inhibits IRES-mediated translation.

Also provided are methods of identifying IRES-containing RNA molecules. The methods comprise inhibiting Rps25 expression or function in a cell, determining a protein expression pattern in the cell, and comparing the protein expression pattern in the cell to a control. A decrease in protein expression of a RNA molecule as compared to a control indicates the RNA molecule contains an IRES.

Further provided are methods of promoting IRES-mediated translation. The method comprises providing a cell, wherein the cell comprises an IRES-containing RNA molecule, and contacting the cell with an agent, wherein the agent increases Rps25 expression or activity in comparison to a control. The method can further comprise determining that IRES-mediated translation is promoted by detecting an increased level of protein expressed by the IRES-containing RNA molecule in comparison to a control.

### DESCRIPTION OF DRAWINGS

FIG. 1 shows a schematic of the secondary structure of the CrPV IGR IRES. The conserved nucleotides across the type I IGR IRESs in the Dicistrovirdae family are capitalized.

FIG. 2 shows S. cerevisiae does not require Rps25 for growth. FIG. 2A shows an image of tetrads that were dissected from heterozygous rps25aΔbΔ diploid yeast, which were sporulated. FIG. 2B (top) shows a map of the image of the yeast growth plate shown in FIG. 2B (bottom). FIG. 2B (bottom) shows an image of a plate demonstrating the growth of wild-type and Rps25 deletion strains with and without the pS25A rescue plasmid on synthetic media. Plates were grown for 3 days at 30° C.

FIG. 3 shows that the CrPV IGR IRES requires Rps25 for translation initiation in vivo. FIG. 3A shows a diagram of the ΔAUG dicistronic luciferase reporter. Transcription of the dicistronic reporter is under the control of the PGK1 promoter. Renilla luciferase is translated by a cap-dependent mechanism, and firefly expression is dependent on a functional IGR IRES. The first AUG of the firefly luciferase coding region has been deleted to ensure that the firefly luciferase activity is solely dependent on a functional IGR IRES, which does not require an AUG start codon for initiation. FIG. 3B shows a histogram representing the IRES activities of wild-type and Rps25 deletion strains with and without the pS25A rescue plasmid transformed with a dicistronic reporter harboring the wild-type (gray bars) or the IGRmut (white bar) IGR IRES. The firefly luciferase values are normalized to Renilla luciferase values as an internal control, and are expressed as a percentage of activity with the CrPV IGR IRES in wild-type yeast arbitrarily set to 100%. Data values are given for each yeast strain, and standard error is indicated for n=3. FIG. 3C shows the Renilla and firefly luciferase values for B; standard error is indicated for n=3.

FIG. 4 shows that the CrPV IGR IRES is unable to bind to 40S ribosomal subunits that lack Rps25. Increasing concentrations of 40S ribosomal subunits from wild-type (top) and rps25aΔbΔ yeast strains with (bottom) and without (middle) the pS25A rescue plasmid were incubated with radiolabeled wild-type CrPV IGR IRES RNA. The asterisk indicates 80S complexes, from contaminating 60S subunits. (Right) The dissociation constant (K_{d}) was determined independently by filter-binding assays. The standard error for n=3 is indicated.

FIG. 5 shows that deletion of Rps25 does not have a significant effect on global translation. FIG. 5A shows polysome analyses of wild-type, rps25aΔ, rps25bΔ, and rps25aΔbΔ deletion strains. Polysome to monosome ratios (P/M) are indicated. FIG. 5B shows a histogram of Protein synthesis rates determined by ³⁵S-methionine incorporation for wild-type and rps25aΔbΔ strains. FIG. 5C shows an image of a gel demonstrating rRNA biogenesis for the wild-type and rps25aΔbΔ strains visualized via pulse-chase labeling with [5,6-³H]uracil. FIG. 5D (top) shows a diagram of the readthrough dual luciferase reporter. Readthrough efficiency was measured for the wild-type, rps25aΔbΔ, and rps25aΔbΔ with the pS25A strains harboring a reporter with a tetranucleotide stop codon as indicated in FIG. 5D (bottom). The fold change between the wild-type and rps25aΔbΔ strains are indicated below each tetranucleotide. Standard error is indicated for n=4. FIG. 5E (top) shows a diagram of the programmed ribosomal frameshifting reporters. Frameshifting efficiencies for each reporter were tested in the following strains: wild-type, rps25aΔbΔ, or rps25aΔbΔ with the pS25A plasmid in FIG. 5E (bottom). Standard error is indicated for n=3. FIG. 5F shows miscoding for the wild-type, rps25aΔbΔ, or rps25aΔbΔ with the rescue plasmid (pS25A) strains in FIG. 5F (bottom). Miscoding was measured using a dual luciferase reporter with a mutation in the firefly ORF shown in FIG. 5F (top). The percent miscoding is indicated above each bar of the graph and standard error is indicated for n=4.

FIG. 6 shows Rps25 is required for CrPV IGR IRES and HCV IRES activities in mammals. FIG. 6A shows an image of a Northern blot demonstrating that Rps25 was knocked down using siRNA. The mRNA levels were examined at 48, 72, and 96 hours after siRNA transfection by Northern analysis. The level of Rps25 mRNA was normalized to β-actin and is expressed as a percentage of the control for each time point. FIG. 6B shows a diagram of the mammalian DNA expression vector containing the CrPV IGR IRES in the ΔAUG dicistronic luciferase reporter. Transcription of the reporter is driven by the CMV promoter. FIG. 6C shows a histogram representing the CrPV IGR IRES activity at 96 hours after siRNA transfection in HeLa cells. Transfection with the reporter plasmid (shown in B) was performed at 48 hours after siRNA knockdown. Standard error is indicated for n=3. FIG. 6D shows the Renilla and firefly luciferase values for FIG. 6C. FIG. 6E shows images of Northern blots demonstrating that Rps25 was knocked down using siRNAs. The mRNA levels were examined by Northern analysis at 72 hours following siRNA transfection. The level of Rps25 mRNA was normalized to β-actin and is expressed as a percentage of the control for each time point. FIG. 6F shows a diagram of the mammalian DNA expression vector containing the HCV IRES in the dicistronic luciferase reporter. FIG. 6G shows a histogram representing the IRES activity of the HCV IRES in cells with either control or Rps25 siRNAs. The reporter was transfected into the cells 24 hours after siRNA knockdown and assayed at 72 hours. Standard error is indicated for n=5 or n=4 for experiments 1 and 2, respectively. FIG. 6H shows the Renilla and firefly luciferase values for FIG. 6G.

FIG. 7 shows that Rps25 is required for CrPV IGR IRES-mediated translation in mammalian cells. FIG. 7A shows a diagram of the discistronic reporter used in mammalian cells. FIG. 7B shows an image of a Northern blot of HeLa cells transduced with a lentivirus containing control of Rps25 shRNA. The Northern blot demonstrates knockdown of Rps25 mRNA levels. The level of Rps25 mRNA was normalized to β-actin and is expressed as a percentage of the control. FIG. 7C shows a histogram representing the CrPV IGR IRES activity after shRNA mediated knockdown of Rps25 in HeLa cells. Standard error is indicated for n=3.

FIG. 8 shows that the decrease in IRES activity is maintained over time. CrPV IGR IRES activity is greatly reduced in stable cell lines expressing shRNA against Rps25. FIG. 8A shows an image of a Northern blot and an image of a Western blot demonstrating knockdown of Rps25 with both siRNAs and shRNAs directed to Rps25. The level of Rps25 mRNA was normalized to β-actin and is expressed as a percentage of the control. FIG. 8B shows a histogram representing the CrPV IGR IRES activity after siRNA and shRNA mediated knockdown of Rps25 in HeLa cells. Standard error is indicated for n=3.

FIG. 9 shows that the Rps25 is required for IRES-mediated translation in both classes of IGR IRESs. The CrPV IRES belongs to a family of viruses called the Dicistroviridae. Additionally, there are two classes of IGR IRESs. The CrPV IRES belongs to class I, and class II members have a larger bulge and an extra stem loop in domain III. As demonstrated in the histogram, each member of the family tested was unable to translate efficiently in the absence of Rps25. Since the depletion of Rps25 affects both classes of IGR IRESs, it is believed that Rps25 interacts with the two stem loops highlighted, as this region is conserved between the two classes.

FIG. 10 shows Rps25 is required for HCV IRES-mediated translation and replication in mammalian cells. FIG. 10A shows an image of a Northern blot demonstrating knockdown of Rps25 with shRNAs directed to Rps25. FIG. 10B shows a histogram representing HCV IRES activity after shRNA mediated knockdown of Rps25 in HeLa cells. Standard error is indicated for n=3. FIG. 10C shows an image of a Western blot demonstrating that HCV replication in Huh7 cells is inhibited by siRNA mediated knockdown of Rps25 (left). Additionally shown is an image of a Northern blot demonstrating that Rps25 mRNA is knocked down by treatment with siRNAs (right). Huh7 cells treated with control or Rps25 siRNA for 24 hours were infected with an HCV replicon. After 72 hours, cells were harvested and protein extracted for quantitative Western analysis using both the β-actin antibody and an antibody to the HCV protein NS5A.

FIG. 11 shows Rps25 enhances the activity of Picornaviral IRESs. Shown is a histogram demonstrating that Rps25 knockdown effects picornaviral IRES-mediated translation. A discistronic reporter harboring one of four viral IRESs was transfected into cells 48 hours after siRNA mediated knockdown of Rps25. IRES activity was measured at 96 hours. Standard error is indicated for n=3. ECMV: Encephalomyocarditis virus; PV: Poliovirus; EV71: Enterovirus 71. The CrPV IGR IRES is shown for comparison.

FIG. 12 shows cellular IRESs demonstrate a moderate to severe dependency on Rps25. FIG. 12A shows an image of a Northern blot demonstrating knockdown of Rps25 with siRNAs directed to Rps25. FIG. 12B shows a histogram representing cellular IRES activity of multiple cellular RNAs known to have IRES elements after siRNA mediated knockdown of Rps25 in HeLa cells. A discistronic reporter assay for cellular IRESs in HeLa cells treated with control and Rps25 siRNA for 48 hours was performed. IRES activity was measured after 96 hours and is expressed as a percentage of the corresponding IRES activity measured in the control cells, which is arbitrarily set to 100%. Standard error is indicated for n=3.

FIG. 13 shows that the Bag-1 cellular IRES requires Rps25 for translation. FIG. 13A shows an image of a Northern blot demonstrating knockdown of Rps25 with siRNAs directed to Rps25. FIG. 13B shows a histogram representing cellular IRES activity of Bag-1 and c-myc after siRNA mediated knockdown of Rps25 in HeLa cells. FIG. 13C shows schematics of stem loops of three IRES elements. IRES elements that depend on Rps25 for translation, the CrPV and Bag-1 IRES elements, share a conserved sequence motif (ANY motif).

FIG. 14 shows a model of the IGR IRES interactions with the 40S ribosome. (Top) The Cryo-EM structure of the IGR IRES bound to a yeast 40S subunit is shown in two orientations. The top left depicts the subunit interface side of the 40S subunit with the IGR IRES bound to the mRNA channel occupying the P and E sites (Schuler et al., Nat. Struct. Mol. Biol. 13:1092-6 (2006)). The top right depicts the complex rotated 90° along the X-axis and 110° along the Y-axis as indicated, to show the backside of SL2.3. Magnifications of the boxed areas show the interactions of SL2.3 and SL2.1 with the 40S subunit. The density of the CrPV IGR IRES has been removed for clarity, and a model of the IGR IRES structure is shown. In addition, atomic models of the prokaryotic rRNA and proteins (PDB:1S1H) have been modeled into the Cryo-EM density (Spahn et al., EMBO J. 23; 1008-19 (2004)). These models reveal an unassigned density at the surface of the ribosome near Rps5 that could be Rps25. A protein at this location would be predicted to interact with the CrPV IGR IRES SL2.3 and may interact with SL2.1 with either an N-terminal or C-terminal extension.

FIG. 15 shows the transient transfection optimization of the HCV IRES dual LUC reporter in Huh7 human liver cells. Multiple cationic lipid-based transient transfection reagents were used to determine if transient transfection of the reporter was feasible for the high-throughput screen.

FIG. 16 shows the high-throughput plate maps for the HCV IRES translation inhibitor screen. FIG. 16 (top) shows the plate map for a semi-automated high-throughput screen. Wells A1-A6 are treated with 1% DMSO in standard culture medium. Wells A7-A12 are transfected with the HCV IRES Dual LUC reporter alone. Wells B1-H12 are treated in triplicate with a test small molecule in triads (e.g., wells B1-B3 are treated with compound A and wells B4-6 are treated with compound B, etc.). FIG. 16 (bottom) shows the plate map for the fully automated high-throughput screen. Column 1 is mock transfected. Columns 2-12 are transfected with HCV IRES Dual LUC reporter. Columns 1 and 12 are not challenged with small molecules but rather have 1% DMSO in standard culture medium. Columns 2-11 are challenged with 80 small molecules plated in a robotic and proprietary triplicate batch deposition format where each small molecule is assessed in three different wells and no two compounds are present together in the same well.

FIG. 17 shows the results of a test run of the high-throughput screen using 960 initial compounds. FIG. 17A shows a histogram demonstrating the ratio of Renilla LUC signal to firefly LUC signal as the degree of HCV virus in control (no test molecules; 1% DMSO in medium) versus experiment conditions (triplicate assessment of test small molecules). An average of 2 hits per plate were observed across 12 microtiter plates. All hit small molecules are shown relative to the mock and transfected controls exposed to 1% DMSO only. FIG. 17B shows a histogram demonstrating the concentration dependent inhibition of HCV IRES-mediated translation using inhibitors at 0.02 to 20 μM. FIG. 17C shows images of Western blots demonstrating that HCV replication is inhibited in Huh7 cells in the presence of 2 μM inhibitor as evidenced by the reduction in NS5A levels after 72 hours post transfection. FIG. 17D shows the initial compounds found to inhibit IRES mediated translation.

FIG. 18 shows a schematic showing a cluster of small molecule hits identified in the high-throughput assay that share commonality in structure and display inhibition of HCV IRES mediated translation in Huh7 human hepatocytes.

FIG. 19 shows additional identified compounds that inhibit IRES mediated translation. FIG. 19A shows a histogram demonstrating the percent IRES activity of test compounds at 2 mM. FIG. 19B shows the structures of the identified compounds that inhibit IRES mediated translation.

### DETAILED DESCRIPTION

Provided herein are compounds for the treatment or prevention of viral infection or cancer in a subject. The viral infection can, for example, be mediated by a virus comprising an internal ribosome entry site (IRES)-containing RNA molecule. The cancer can, for example, be caused by an increased or decreased IRES-mediated translation of a cellular mRNA molecule.

The compounds for the treatment of viral infections (e.g., HCV) or cancer (e.g., breast cancer) as described herein include compounds represented by Formula I:

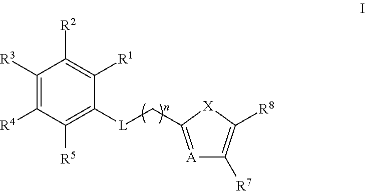

and pharmaceutically acceptable salt of prodrug thereof.

In Formula I, A is CR⁹ or N. In some examples, A is CH or N.

Also, in Formula I, L is —O—CR¹⁰R¹¹C(O)—NR⁶—, —NR¹²—NR⁶—, —C(O)—NR⁶—, —SO₂—NR⁶—, —CH₂—NR⁶—, —CH₂—CH₂—NR⁶—, or a substituted or unsubstituted heteroaryl. In some examples, L is a substituted or unsubstituted pyrazole.

Additionally, in Formula I, n is 0, 1, or 2.

Also, in Formula I, X is —CR¹³═CR¹⁴—, —N═CR¹⁵—, —CR¹⁵═N—, NR¹⁶, O, or S. X can be an atom in a five-membered ring or a six-membered ring. For example, when X is NR¹⁶, O, or S, X is an atom of a five-membered ring (e.g., thiophenyl, pyrrolyl, furanyl, oxazolyl, thiazolyl, or imidazolyl). When X is —CR¹³═CR¹⁴—, —N═CR¹⁵—, or —CR¹⁵═N—, X is an atom of a six-membered ring, such as, for example, phenyl, pyridinyl, or pyrazinyl. In some examples, X is S or —CH═CH—.

Further in Formula I, R¹, R², R³, R⁴, R⁵, R⁷, R⁸, R⁹, R¹⁰, R¹¹, R¹³, R¹⁴, and R¹⁵ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl. In some examples, R³ is ethoxy, dimethylamino, or chloro.

Also, in Formula I, R⁶, R¹², and R¹⁶ are each independently selected from hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl, or substituted or unsubstituted carbonyl.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, pyrazole, and pyrazine. Aryl and heteroaryl molecules can also include additional fused rings, for example, benzofuran, indole, benzothiophene, naphthalene, anthracene, and quinoline.

The alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl molecules used herein can be substituted or unsubstituted. As used herein, the term substituted includes the addition of an alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl group to a position attached to the main chain of the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl, e.g., the replacement of a hydrogen by one of these molecules. Examples of substitution groups include, but are not limited to, hydroxyl, halogen (e.g., F, Br, Cl, or I), and carboxyl groups. Conversely, as used herein, the term unsubstituted indicates the alkyl, alkenyl, alkynyl, aryl, heteroalkyl, heteroalkenyl, heteroalkynyl, heteroaryl, cycloalkyl, cycloalkenyl, cycloalkynyl, heterocycloalkyl, heterocycloalkenyl, or heterocycloalkynyl has a full compliment of hydrogens, i.e., commensurate with its saturation level, with no substitutions, e.g., linear decane (—(CH₂)₉—CH₃).

In Compound I, adjacent R groups on the phenyl ring, i.e., R¹, R², R³, R⁴, and R⁵, can be combined to form substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl groups. For example, R⁵ can be a formamide group and R⁶ can be an ethylene group that combine to form a pyridinone group. Other adjacent R groups include the combinations of R¹ and R², R² and R³, and R³ and R⁴.

Specific examples of Formula I are as follows:

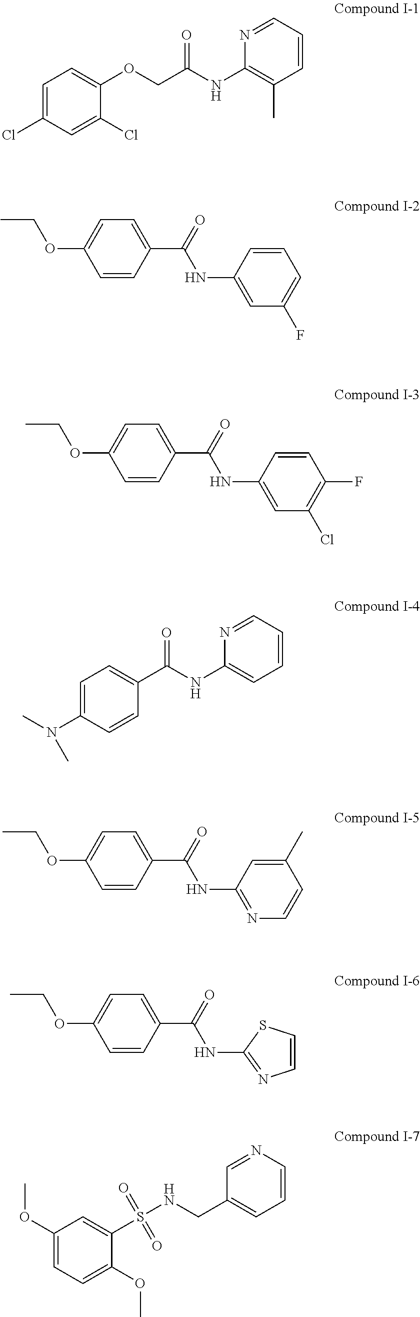

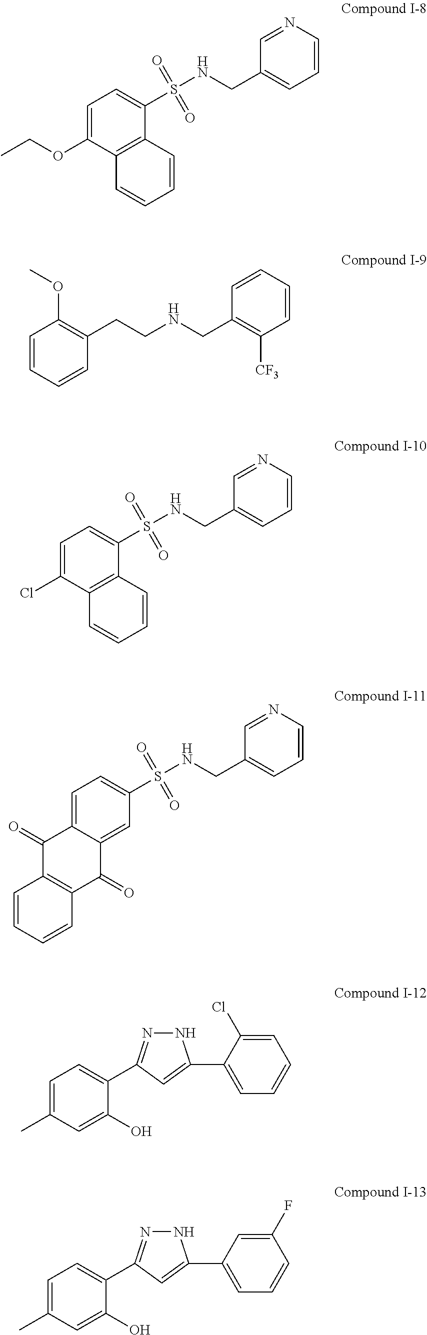

Variations on the Formula I include the addition, subtraction, or movement of the various constituents as described for each compound. Similarly, when one or more chiral centers are present in a molecule, the chirality of the molecule can be changed. The compounds described herein can be isolated in pure form or as a mixture of isomers. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed., Wiley & Sons, 2006, which is incorporated herein by reference in its entirety.

The compounds described herein can be prepared in a variety of ways known to one skilled in the art of organic synthesis or variations thereon as appreciated by those skilled in the art. The compounds described herein can be prepared from readily available starting materials. Optimum reaction conditions may vary with the particular reactants or solvents used, but such conditions can be determined by one skilled in the art.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

Provided herein are methods of treating or preventing a viral infection in a subject. The methods comprise identifying a subject with or at risk of developing a viral infection, wherein the viral infection is mediated by a virus comprising an IRES-containing RNA molecule and administering to the subject a therapeutically effective amount of any of the compounds disclosed herein. The compounds can, for example, reduce Rps25 expression or function in the subject in comparison to a control. Optionally, the methods further comprise administering to the subject a therapeutically effective amount of an agent that reduces Rps25 expression or function in the subject in comparison to a control.

The methods can, for example, comprise identifying a subject with or at risk of developing a viral infection, wherein the viral infection is mediated by a virus comprising an IRES-containing RNA molecule and administering to the subject a therapeutically effective amount of an agent that reduces Rps25 expression or function in the subject in comparison to a control.

As used throughout, the agent that reduces Rps25 expression or function can, for example, be selected from the group consisting of a small molecule, a polypeptide, a nucleic acid molecule, a peptidomimetic, or a combination thereof. Optionally, the nucleic acid molecule is selected from the group consisting of an antisense molecule, a short-interfering RNA (siRNA) molecule, a microRNA (miRNA) molecule, a RNA aptamer, or a combination thereof. The siRNA molecule can, for example, comprise SEQ ID NO:5.

Optionally, the virus is selected from the group consisting of a virus within the Picornaviridae Family, a virus within the Dicistroviridae Family, a virus within the Flaviviridae Family, a virus within the Herpesviridae Family, a virus within the Retroviridae Family, and a virus within the Poxviridae Family. Optionally, the virus is selected from the group consisting of a cricket paralysis virus, a taura syndrome virus, and an Israel acute paralysis virus. Optionally, the virus is hepatitis C virus (HCV).

Also provided herein is a method of inhibiting internal ribosome entry site (IRES)-mediated translation. The method comprises providing a cell, wherein the cell comprises an IRES-containing RNA molecule and contacting the cell with an agent that reduces Rps25 expression or function. Reduction of Rps25 expression or function as compared to a control indicates the agent inhibits IRES-mediated translation. Optionally, the method further comprises determining that IRES-mediated translation is inhibited by determining a reduced level of protein expressed by the IRES-containing RNA molecule in comparison to a control. The expression of Rps25 can be reduced by decreasing the level of Rps25 RNA or protein expression. The function of Rps25 can, for example, be reduced by blocking binding of Rps25 to the IRES-containing RNA molecule. Optionally, the function of Rps25 can be reduced by blocking binding of Rps25 to the 40S ribosomal subunit.

Optionally, the IRES-containing mRNA is selected from the group consisting of a firefly luciferase mRNA, a VEGF mRNA, a MNT mRNA, a Set7 mRNA, a L-myc mRNA, a MTG8a mRNA, a Myb mRNA, a BIP mRNA, an eIF4G mRNA, a PIM-1 mRNA, a CYR61 mRNA, a p27 mRNA, a XIAP mRNA, a BAG-1 mRNA, or a combination thereof.

Further provided are methods of treating or preventing cancer in a subject. The methods comprise identifying a subject with or at risk of developing cancer, wherein the cancer is related to increased or decreased IRES-mediated translation of an mRNA molecule, and administering to the subject a therapeutically effective amount of any of the compounds described herein. Optionally, the compound reduces Rps25 expression or function in the subject in a cancer related to increased IRES-mediated translation of an mRNA. Optionally, the method further comprises administering to the subject a therapeutically effective amount of an agent that reduces Rps25 expression or function in comparison to a control in a cancer related to increased IRES-mediated translation of an mRNA. Optionally, the compound increases Rps25 expression or function in a cancer related to decreased IRES-mediated translation of an mRNA. Optionally, the method further comprises administering to the subject a therapeutically effective amount of an agent that increases Rps25 expression or function in comparison to a control in a cancer related to decreased IRES-mediated translation of an mRNA.

The methods can, for example, comprise identifying a subject with or at risk of developing cancer, wherein the cancer is related to increased IRES-mediated translation of an mRNA molecule, and administering to the subject a therapeutically effective amount of an agent that reduces Rps25 expression or function in comparison to a control. The methods can, for example, comprise identifying a subject with or at risk of developing cancer, wherein the cancer is related to decreased IRES-mediated translation of an mRNA molecule, and administering to the subject a therapeutically effective amount of an agent that increases Rps25 expression or function in comparison to a control. Optionally, the agent is a nucleic acid molecule. The nucleic acid molecule can, for example, comprise a nucleic acid encoding a Rps25 or a functional fragment thereof.

As defined herein, a cancer related to increased or decreased IRES-mediated translation is a cancer caused by, a cancer that metastasizes due to, and/or a cancer present that exhibits an increase or decrease in translation of one or more IRES containing mRNAs. The increase or decrease in translation of one or more IRES containing mRNAs directly or indirectly contributes to any timepoint in the lifespan of the cancer, from the birth of the cancer through the metastasis of the cancer. Examples of cancers include, but are not limited to, breast cancer, prostate cancer, lung cancer, liver cancer, pancreatic cancer, skin cancer, testicular cancer, ovarian cancer, thyroid cancer, mouth/esophageal cancer, and/or brain cancer.

Also provided is a method of screening for an agent that inhibits or promotes IRES-mediated translation. The method comprises providing a system comprising a Rps25 or a nucleic acid that encodes Rps25 and an IRES-containing RNA molecule, contacting the system with the agent to be screened, and determining Rps25 expression or function. A decrease in the level of Rps25 expression or function indicates the agent inhibits IRES-mediated translation. An increase in the level of Rps25 expression or function indicates the agent promotes IRES-mediate translation. Optionally, the system comprises a cell. The cell can contain naturally occurring IRES-containing RNA molecules. The cell can also be modified to contain artificial IRES-containing RNA molecules. Optionally, the system comprises an in vitro assay. The agent to be tested can, for example, be selected from the group consisting of a small molecule, a polypeptide, a nucleic acid molecule, a peptidomimetic, or a combination thereof. Also provided are agents isolated by the methods of screening described herein.

Also provided is a method of identifying IRES-containing RNA molecules. The methods comprise inhibiting Rps25 expression or function in a cell, determining a protein expression pattern in the cell; and comparing the protein expression pattern to a control. A decrease in protein expression of an RNA molecule as compared to a control indicates the RNA molecule contains an IRES. The methods can comprise identifying a novel IRES-containing RNA molecule or verifying a previously hypothesized IRES-containing RNA molecule. Rps25 expression of function can be inhibited using the agents described herein, e.g., the siRNA comprising SEQ ID NO:5. Determining the protein expression pattern of a cell can, for example, comprise doing a protein array or performing a deep sequencing assay on polysomal fractions within the cell. Alternatively, determining the protein expression pattern can comprise using other methods of determining protein expression known in the art.

Further provided is a method of promoting IRES-mediated translation, the method comprising providing a cell, wherein the cell comprises an IRES-containing RNA molecule and contacting the cell with an agent that increases Rps25 expression or function in comparison to a control. An increase in Rps25 expression or function indicates the agent promotes IRES-mediated translation. Optionally, the method further comprises determining that IRES-mediated translation is promoted by detecting an increased level of protein encoded by the IRES-containing RNA molecule in comparison to a control.

Also provided is a method of promoting IRES-mediated translation, the method comprising providing a cell with a nucleic acid encoding a Rps25 protein or a functional fragment thereof. Such a method can be in vivo or in vitro.

Also provided is method of detecting cancer in a subject, the method comprising determining the levels of Rps25 expression in a subject, comparing the levels of Rps25 to a standard, and determining the presence of cancer. A modulation in the level of Rps25 translation or function correlates with the presence of cancer. Similar steps can be used to detect the effectiveness of treatment. For example, levels of Rps25 are detected and an increase in the level of Rps25 translation or function indicates the treatment is ineffective or in need of change.

As described herein, an IRES-containing RNA molecule can be artificially created or naturally occurring. An artificially created IRES-containing RNA molecule can, for example, be a firefly luciferase mRNA that contains an IRES controlling translation of the firefly luciferase protein. An artificially created IRES-containing RNA molecule can also be a green fluorescent protein mRNA that contains an IRES controlling translation of the green fluorescent protein. These IRES-containing RNA molecules are generally used as reporters for IRES-mediated translation. A naturally occurring IRES-containing RNA molecule can, for example, be a cellular or a viral RNA molecule. An IRES-containing cellular RNA can for example, be selected from the group consisting of a VEGF mRNA, a MNT mRNA, a Set7 mRNA, a L-myc mRNA, a MTG8a mRNA, a Myb mRNA, a BIP mRNA, an eIF4G mRNA, a PIM-1 mRNA, a CYR61 mRNA, a p27 mRNA, a XIAP mRNA, and a BAG-1 mRNA. An IRES-containing viral mRNA molecule can be found in viruses of the Picornaviridae Family, viruses of the Dicistroviridae Family, viruses of the Flaviviridae Family, viruses of the Retroviridae Family, viruses in the Herpesviridae Family, or in viruses in the Poxviridae Family.

As described herein, the level of Rps25 protein expression can, for example, be determined using an assay selected from the group consisting of Western blot, enzyme-linked immunosorbent assay (ELISA), enzyme immunoassay (EIA), radioimmunoassay (RIA), or protein array. The level of Rps25 RNA expression can, for example, be determined using an assay selected from the group consisting of microarray analysis, gene chip, Northern blot, in situ hybridization, reverse transcription-polymerase chain reaction (RT-PCR), one step PCR, and real-time quantitative real time (qRT)-PCR. The analytical techniques to determine protein or RNA expression are known. See, e.g., Sambrook et al., Molecular Cloning: A Laboratory Manual, 3rd Ed., Cold Spring Harbor Press, Cold Spring Harbor, N.Y. (2001).

As described herein, the level of Rps25 function can, for example, be determined by using an assay selected from the group consisting of an RNA mobility shift assay, an RNA crosslinking assay, an RNA affinity assay, a protein-protein binding assay, and an assay measuring IRES-mediated translation of an IRES-containing RNA molecule. A decrease in Rps25 function can, for example, be demonstrated by a loss of binding to an IRES-containing RNA molecule, a loss of binding to the 40S ribosomal subunit, or a decrease in IRES-mediated translation of an IRES-containing RNA molecule as compared to a control. An increase in Rps25 function can, for example, be demonstrated by an enhanced binding to an IRES-containing RNA molecule, an enhanced binding to the 40S ribosomal subunit, or an increase in IRES-mediated translation of an IRES-containing RNA molecule as compared to a control. An increase in Rps25 function can also be demonstrated by an increase in IRES-mediated translation of an IRES-containing molecule in comparison to a control.

As used herein an agent can, for example, be selected from the group consisting of a small molecule, a polypeptide, a nucleic acid molecule, a peptidomimetic, or a combination thereof. Optionally, the polypeptide is an antibody (e.g., to Rps25, to the 40S ribosomal subunit, or to the IRES itself). Optionally, the nucleic acid molecule is an Rps25 inhibitory nucleic acid molecule.

An Rps25 inhibitory nucleic acid molecule can, for example, be selected from the group consisting of an antisense molecule, a short-interfering RNA (siRNA) molecule, a microRNA (miRNA) molecule, a RNA aptamer, or a combination thereof.

A 21-25 nucleotide siRNA or miRNA sequence can, for example, be produced from an expression vector by transcription of a short-hairpin RNA (shRNA) sequence, a 60-80 nucleotide precursor sequence, which is subsequently processed by the cellular RNAi machinery to produce either an siRNA or miRNA sequence. Alternatively, a 21-25 nucleotide siRNA or miRNA sequence can, for example, be synthesized chemically. Chemical synthesis of siRNA or miRNA sequences is commercially available from such corporations as Dharmacon, Inc. (Lafayette, Colo.), Qiagen (Valencia, Calif.), and Ambion (Austin, Tex.). A siRNA sequence preferably binds a unique sequence within the Rps25 mRNA with exact complementarity and results in the degradation of the Rps25 mRNA molecule. A siRNA sequence can bind anywhere within the Rps25 mRNA molecule. Optionally, the Rps25 siRNA sequence can target the sequence 5′-GGACUUAUCAAAC UGGUUU-3′ (SEQ ID NO:11), corresponding to nucleotides 283-301 of the human Rps25 mRNA nucleotide sequence, wherein position 1 begins with the first nucleotide of the coding sequence of the Rps25 mRNA molecule at Accession Number NM_{—}001028 on GenBank. Optionally, the siRNA sequence comprises SEQ ID NO:5. A miRNA sequence preferably binds a unique sequence within the Rps25 mRNA with exact or less than exact complementarity and results in the translational repression of the Rps25 mRNA molecule. A miRNA sequence can bind anywhere within the Rps25 mRNA sequence, but preferably binds within the 3′ untranslated region of the Rps25 mRNA molecule. Methods of delivering siRNA or miRNA molecules are known in the art, e.g., see Oh and Park, Adv. Drug. Deliv. Rev. 61(10):850-62 (2009); Gondi and Rao, J. Cell Physiol. 220(2):285-91 (2009); and Whitehead et al., Nat. Rev. Drug Discov. 8(2):129-38 (2009).

Antisense nucleic acid sequences can, for example, be transcribed from an expression vector to produce an RNA which is complementary to at least a unique portion of the Rps25 mRNA and/or the endogenous gene which encodes Rps25. Hybridization of an antisense nucleic acid under specific cellular conditions results in inhibition of Rps25 protein expression by inhibiting transcription and/or translation.

Antibodies described herein bind the Rps25 and antagonize the function of the Rps25. Optionally, the antibodies described herein bind IRES elements and inhibit the binding of Rps25 to the IRES element. The term antibody is used herein in a broad sense and includes both polyclonal and monoclonal antibodies. The term can also refer to a human antibody and/or a humanized antibody. Examples of techniques for human monoclonal antibody production include those described by Cole et al. (Monoclonal Antibodies and Cancer Therapy, Alan R. Liss, p. 77, 1985) and by Boerner et al. (J. Immunol. 147(1):86-95 (1991)). Human antibodies (and fragments thereof) can also be produced using phage display libraries (Hoogenboom et al., J. Mol. Biol. 227:381 (1991); Marks et al., J. Mol. Biol. 222:581 (1991)). The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA 90:2551-5 (1993); Jakobovits et al., Nature 362:255-8 (1993); Bruggermann et al., Year in Immunol. 7:33 (1993)).

As used herein, the term antibody encompasses, but is not limited to, whole immunoglobulin (i.e., an intact antibody) of any class. The term antibody or fragments thereof can also encompass chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are provided. For example, fragments of antibodies which maintain Rps25 and/or IRES binding activity are included within the meaning of the term antibody or fragment thereof.

Optionally, the antibody is a monoclonal antibody. The term monoclonal antibody as used herein refers to an antibody from a substantially homogeneous population of antibodies, i.e., the individual antibodies comprising the population are identical except for possible naturally occurring mutations that may be present in minor amounts. Monoclonal antibodies may be prepared using hybridoma methods, such as those described by Kohler and Milstein, Nature, 256:495 (1975) or Harlow and Lane, Antibodies, A Laboratory Manual. Cold Spring Harbor Publications, New York (1988). The monoclonal antibodies may also be made by recombinant DNA methods, such as those described in U.S. Pat. No. 4,816,567.

Optionally, Rps25 is human. Optionally, Rps25 is non-human (e.g., rodent, canine, or feline). There are a variety of sequences that are disclosed on Genbank, and these sequences and others are herein incorporated by reference in their entireties as are individual subsequences or fragments contained therein. As used herein, Rps25 refers to the ribosomal S25 polypeptide and homologs, variants, and isoforms thereof. For example, the nucleotide and amino acid sequences of human Rps25 be found at GenBank Accession Nos. NM_{—}001028 and NP_{—}001019.1, respectively. Thus provided is the nucleotide sequence of Rps25 comprising a nucleotide sequence at least about 70%, 75%, 80%, 85%, 90%, 95%, 98%, 99% or more identical to the nucleotide sequence of the aforementioned GenBank Accession Number. Also provided is the amino acid sequence of Rps25 comprising an amino acid sequence at least about 70%, 75%, 80%, 85%, 90%, 95%, 98%, 99% or more identical to the sequence of the aforementioned GenBank Accession Number.

Nucleic acids that encode the polypeptide sequences, variants, and fragments thereof are disclosed. These sequences include all degenerate sequences related to a specific protein sequence, i.e., all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequences.

As used herein, the term peptide, polypeptide or protein is used to mean a molecule comprised of two or more amino acids linked by a peptide bond. Protein, peptide, and polypeptide are also used herein interchangeably to refer to amino acid sequences. It should be recognized that the term polypeptide or protein is not used herein to suggest a particular size or number of amino acids comprising the molecule and that a polypeptide of the disclosure can contain up to several amino acid residues or more.

As with all peptides, polypeptides, and proteins, including fragments thereof, it is understood that additional modifications in the amino acid sequence of the variant Rps25 polypeptides can occur that do not alter the nature or function of the peptides, polypeptides, or proteins. Such modifications include conservative amino acids substitutions and are discussed in greater detail below.

The polypeptides provided herein have a desired function. Rps25 is part of a ribosomal complex that binds IRES elements and promotes IRES-mediated translation. The polypeptides are tested for their desired activity using the in vitro assays described herein.

The polypeptides described herein can be further modified and varied so long as the desired function is maintained. It is understood that one way to define any known modifications and derivatives or those that might arise, of the disclosed genes and proteins herein is through defining the modifications and derivatives in terms of identity to specific known sequences. Specifically disclosed are polypeptides which have at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to Rps25 and variants provided herein. Those of skill in the art readily understand how to determine the identity of two polypeptides. For example, the identity can be calculated after aligning the two sequences so that the identity is at its highest level.

Another way of calculating identity can be performed by published algorithms. Optimal alignment of sequences for comparison may be conducted by the local identity algorithm of Smith and Waterman, Adv. Appl. Math 2:482 (1981), by the identity alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of identity can be obtained for nucleic acids by, for example, the algorithms disclosed in Zuker, Science 244:48-52 (1989); Jaeger et al., Proc. Natl. Acad. Sci. USA 86:7706-10 (1989); Jaeger et al., Methods Enzymol. 183:281-306 (1989), which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity and to be disclosed herein.

Protein modifications include amino acid sequence modifications. Modifications in amino acid sequence may arise naturally as allelic variations (e.g., due to genetic polymorphism), may arise due to environmental influence (e.g., by exposure to ultraviolet light), or may be produced by human intervention (e.g., by mutagenesis of cloned DNA sequences), such as induced point, deletion, insertion, and substitution mutants. These modifications can result in changes in the amino acid sequence, provide silent mutations, modify a restriction site, or provide other specific mutations. Amino acid sequence modifications typically fall into one or more of three classes: substitutional, insertional, or deletional modifications. Insertions include amino and/or terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional modifications are those in which at lease one residue has been removed and a different residues inserted in its place. Such substitutions generally are made in accordance with the following Table 1 and are referred to as conservative substitutions.

TABLE 1
Amino Acid Substitutions

| Amino Acid | Substitutions (others are known in the art) |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

Modifications, including the specific amino acid substitutions, are made by known methods. By way of example, modifications are made by site specific mutagenesis of nucleotides in the DNA encoding the protein, thereby producing DNA encoding the modification, and thereafter expressing the DNA in recombinant cell culture. Techniques for making substitution mutations at predetermined sites in DNA having a known sequence are well known, for example Ml3 primer mutagenesis and PCR mutagenesis.

Provided herein are methods of treating or preventing viral infection or cancer in a subject. Such methods include administering an effective amount of the compounds disclosed herein or an agent comprising a small molecule, a polypeptide, a nucleic acid molecule, a peptidomimetic or a combination thereof. Optionally, the small molecules, polypeptides, nucleic acid molecules, and/or peptidomimetics are contained within a pharmaceutical composition.

Provided herein are compositions containing the provided small molecules, polypeptides, nucleic acid molecules, and/or peptidomimetics, optimally with a pharmaceutically acceptable carrier described herein. The herein provided compositions are suitable for administration in vitro or in vivo. By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. The carrier is selected to minimize any degradation of the active ingredient and to minimize any adverse side effects in the subject.

Suitable carriers and their formulations are described in Remington: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally about 5 to about 8 or from about 7 to 7.5. Other carriers include sustained release preparations such as semipermeable matrices of solid hydrophobic polymers containing the immunogenic polypeptides. Matrices are in the form of shaped articles, e.g., films, liposomes, or microparticles. Certain carriers may be more preferable depending upon, for instance, the route of administration and concentration of composition being administered. Carriers are those suitable for administration of the agent, e.g., the small molecule, polypeptide, nucleic acid molecule, and/or peptidomimetic, to humans or other subjects.

The compositions are administered in a number of ways depending on whether local or systemic treatment is desired, and on the area to be treated. The compositions are administered via any of several routes of administration, including topically, orally, parenterally, intravenously, intra-articularly, intraperitoneally, intramuscularly, subcutaneously, intracavity, transdermally, intrahepatically, intracranially, nebulization/inhalation, or by installation via bronchoscopy. Optionally, the composition is administered by oral inhalation, nasal inhalation, or intranasal mucosal administration. Administration of the compositions by inhalant can be through the nose or mouth via delivery by spraying or droplet mechanism. For example, in the form of an aerosol. In the case of cancer treatment, the composition or agent can be administered directly into or onto a tumor.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives are optionally present such as, for example, antimicrobials, anti-oxidants, chelating agents, and inert gases and the like.

Formulations for topical administration include ointments, lotions, creams, gels, drops, suppositories, sprays, liquids, and powders. Conventional pharmaceutical carriers, aqueous, powder, or oily bases, thickeners and the like are optionally necessary or desirable.

Compositions for oral administration include powders or granules, suspension or solutions in water or non-aqueous media, capsules, sachets, or tables. Thickeners, flavorings, diluents, emulsifiers, dispersing aids or binders are optionally desirable.

Optionally, the nucleic acid molecule or polypeptide is administered by a vector comprising the nucleic acid molecule or a nucleic acid sequence encoding the polypeptide. There are a number of compositions and methods which can be used to deliver the nucleic acid molecules and/or polypeptides to cells, either in vitro or in vivo via, for example, expression vectors. These methods and compositions can largely be broken down into two classes: viral based delivery systems and non-viral based deliver systems. Such methods are well known in the art and readily adaptable for use with the compositions and methods described herein.

As used herein, plasmid or viral vectors are agents that transport the disclosed nucleic acids into the cell without degradation and include a promoter yielding expression of the nucleic acid molecule and/or polypeptide in the cells into which it is delivered. Viral vectors are, for example, Adenovirus, Adeno-associated virus, herpes virus, Vaccinia virus, Polio virus, Sindbis, and other RNA viruses, including these viruses with the HIV backbone. Also preferred are any viral families which share the properties of these viruses, which make them suitable for use as vectors. Retroviral vectors, in general are described by Coffin et al., Retroviruses, Cold Spring Harbor Laboratory Press (1997), which is incorporated by reference herein for the vectors and methods of making them. The construction of replication-defective adenoviruses has been described (Berkner et al., J. Virology 61:1213-20 (1987); Massie et al., Mol. Cell. Biol. 6:2872-83 (1986); Haj-Ahmad et al., J. Virology 57:267-74 (1986); Davidson et al., J. Virology 61:1226-39 (1987); Zhang et al., BioTechniques 15:868-72 (1993)). The benefit and the use of these viruses as vectors is that they are limited in the extent to which they can spread to other cell types, since they can replicate within an initial infected cell, but are unable to form new infections viral particles. Recombinant adenoviruses have been shown to achieve high efficiency after direct, in vivo delivery to airway epithelium, hepatocytes, vascular endothelium, CNS parenchyma, and a number of other tissue sites. Other useful systems include, for example, replicating and host-restricted non-replicating vaccinia virus vectors.

The provided polypeptides and/or nucleic acid molecules can be delivered via virus like particles. Virus like particles (VLPs) consist of viral protein(s) derived from the structural proteins of a virus. Methods for making and using virus like particles are described in, for example, Garcea and Gissmann, Current Opinion in Biotechnology 15:513-7 (2004).

The provided polypeptides can be delivered by subviral dense bodies (DBs). DBs transport proteins into target cells by membrane fusion. Methods for making and using DBs are described in, for example, Pepperl-Klindworth et al., Gene Therapy 10:278-84 (2003).

The provided polypeptides can be delivered by tegument aggregates. Methods for making and using tegument aggregates are described in International Publication No. WO 2006/110728.

Non-viral based delivery methods can include expression vectors comprising nucleic acid molecules and nucleic acid sequences encoding polypeptides, wherein the nucleic acids are operably linked to an expression control sequence. Suitable vector backbones include, for example, those routinely used in the art such as plasmids, artificial chromosomes, BACs, YACs, or PACs. Numerous vectors and expression systems are commercially available from such corporations as Novagen (Madison, Wis.), Clonetech (Palo Alto, Calif.), Stratagene (La Jolla, Calif.), and Invitrogen/Life Technologies (Carlsbad, Calif.). Vectors typically contain one or more regulatory regions. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

Preferred promoters controlling transcription from vectors in mammalian host cells may be obtained from various sources, for example, the genomes of viruses such as polyoma, Simian Virus 40 (SV40), adenovirus, retroviruses, hepatitis B virus, and cytomegalovirus (CMV), or from heterologous mammalian promoters, e.g. β-actin promoter or EF1α promoter, or from hybrid or chimeric promoters (e.g., CMV promoter fused to the β-actin promoter). Of course, promoters from the host cell or related species are also useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 bp in length, and they function in cis. Enhancers usually function to increase transcription from nearby promoters. Enhancers can also contain response elements that mediate the regulation of transcription. While many enhancer sequences are known from mammalian genes (globin, elastase, albumin, fetoprotein, and insulin), typically one will use an enhancer from a eukaryotic cell virus for general expression. Preferred examples are the SV40 enhancer on the late side of the replication origin, the cytomegalovirus early promoter enhancer, the polyoma enhancer on the late side of the replication origin, and adenovirus enhancers.

The promoter and/or the enhancer can be inducible (e.g. chemically or physically regulated). A chemically regulated promoter and/or enhancer can, for example, be regulated by the presence of alcohol, tetracycline, a steroid, or a metal. A physically regulated promoter and/or enhancer can, for example, be regulated by environmental factors, such as temperature and light. Optionally, the promoter and/or enhancer region can act as a constitutive promoter and/or enhancer to maximize the expression of the region of the transcription unit to be transcribed. In certain vectors, the promoter and/or enhancer region can be active in a cell type specific manner. Optionally, in certain vectors, the promoter and/or enhancer region can be active in all eukaryotic cells, independent of cell type. Preferred promoters of this type are the CMV promoter, the SV40 promoter, the β-actin promoter, the EF1α promoter, and the retroviral long terminal repeat (LTR).

The vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, Conn.) sequences typically are expressed as a fusion with the encoded polypeptide. Such tags can be inserted anywhere within the polypeptide including at either the carboxyl or amino terminus.

As used throughout, subject can be a vertebrate, more specifically a mammal (e.g. a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient or subject may be used interchangeably and can refer to a subject with a disease or disorder (e.g. viral infection or cancer). The term patient or subject includes human and veterinary subjects.

Subjects include those with or at risk of developing cancer or with or at risk of viral infection. A subject at risk of developing cancer can be genetically predisposed to the cancer, e.g., have a family history or have a mutation in a gene that causes the disease or disorder or may be immunocompromised. A subject at risk of developing a viral infection can be predisposed to the viral infection, e.g., have an occupation putting the subject at risk for contracting a viral infection, have a compromised immune system, or have been exposed to a virus. A subject currently with cancer or a viral infection has one or more than one symptom of cancer or the viral infection and may have been diagnosed with cancer or the viral infection.

The methods and agents as described herein are useful for both prophylactic and therapeutic treatment. For prophylactic use, a therapeutically effective amount of the agent described herein is administered to a subject prior to onset (e.g., before obvious signs of cancer or a viral infection) or during early onset (e.g., upon initial signs and symptoms of cancer or a viral infection). Prophylactic administration can occur for several days to years prior to the manifestation of symptoms of cancer or a viral infection. Prophylactic administration can be used, for example, in the preventative treatment of subjects diagnosed with a genetic predisposition to cancer. Therapeutic treatment involves administering to a subject a therapeutically effective amount of the agents described herein after diagnosis or development of cancer or a viral infection.

According to the methods taught herein, the subject is administered an effective amount of the agent. The terms effective amount and effective dosage are used interchangeably. The term effective amount is defined as any amount necessary to produce a desired physiologic response (e.g., a decrease in the level of IRES mediated translation resulting in the treatment of a viral infection or a cancer). Effective amounts and schedules for administering the agent may be determined empirically, and making such determinations is within the skill in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary, and can be administered in one or more dose administrations daily, for one or several days. Guidance can be found in the literature for appropriate dosages for given classes of pharmaceutical products.

As used herein the terms treatment, treat, or treating refers to a method of reducing the effects of a disease (e.g., cancer) or condition (e.g., viral infection) or symptom of the disease or condition. Thus in the disclosed method, treatment can refer to a 10%, 20%, 30%, 40%, 50%, 60%, 70%, 80%, 90%, or 100% reduction in the severity of an established disease or condition or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. Treatment can also include a delay in the progression of one or more symptoms. It is understood that treatment does not necessarily refer to a cure or complete ablation of the disease, condition, or symptoms of the disease or condition. Thus, treatment refers, for example, to an improvement in one or more symptoms of a viral infection or a cancer.

As used herein, the terms prevent, preventing, and prevention of a disease (e.g., cancer) or condition (e.g., viral infection) refers to an action, for example, administration of a therapeutic agent, that occurs before or at about the same time a subject begins to show one or more symptoms of the disease or condition, which inhibits or delays onset or exacerbation of one or more symptoms of the disease or condition. As used herein, references to decreasing, reducing, or inhibiting include a change of 10%, 20%, 30%, 40%, 50%, 60%, 70%, 80%, 90% or greater as compared to a control level. Such terms can include but do not necessarily include complete elimination.

By control is meant in the absence of treatment or in the absence of an agent or composition. Thus, a control can be a known standard, or the subject, cell, or system before or after treatment. A control can also be an untreated subject, cell, or system.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

### EXAMPLES

### General Methods

### General Yeast and Cell Culture

S. cerevisiae strains used in this study were from the Saccharomyces deletion project: wild-type (BY4741: MATα his3Δ1 leu2Δ0 met15Δ0 ura3Δ0), rps25aΔ (BY4657: MATα his3Δ1 leu2Δ0 met15Δ0 ura3Δ0 rps25a::KanMX), and rps25bD (BY15242: MATα his3Δ1 leu2Δ0 lys2Δ0 ura3Δ0 rps25b::KanMX) (Winzeler et al., Science 285:901-6 (1999)). rps25aΔbΔ (SRT221: MATα his3Δ1 leu2Δ0 lys2Δ0 ura3Δ0 rps25a::anMX rps25b::KanMX) was generated by mating BY4657 and BY15242, sporulating, and dissecting the tetrads using standard genetic techniques (Treco and Winston, Curr. Protoc. Mol. Biol. 82; 13.12.11-13.12.12 (2008)). Standard methods were used to grow and transform yeast strains (Becker and Lundblad, Curr. Protoc. Mol. Biol. 27:13.17.11-13.17.10 (1993); Treco and Lundblad, Curr. Protoc. Mol. Biol. 23:13.11.11-13.11.17 (1993)). A Southern blot was performed to confirm that both RPS25A and RPS25B are disrupted in the rps25aΔbΔ yeast strain.

HeLa cells (Ambion; Austin, Tex.) were maintained in complete media (high-glucose Dulbecco's modified Eagle's medium [DMEM] supplemented with 10% [v/v] fetal calf serum, 1% [v/v] L-glutamine, 1% [v/v] penicillin and streptomycin) at 37° C. and 5% CO2.

### Plasmid Manipulations

A UAA stop codon was inserted into the pS25A rescue plasmid (Open Biosystems; Huntsvilled, Ala., catalog no. YSC3869-9518490) following the RPS25A ORF and before the C-terminal His6 tag by site-directed mutagenesis, as described previously (Deniz et al., RNA 15:932-46 (2009)), using primers (S25addstop_sense, 5′-AACCACTTTGTACAAGAAAGCTTAGTTTTCAGAAGCAGTAGCTCTG-3′ (SEQ ID NO:1); S25addstop_antisense, 5′-CAGAGCTACTGCTTCTGAAAACTAAGCTTTCTT GTACAAAGTGGTT-3′ (SEQ ID NO:2). To generate a high-copy dicistronic reporter (pSRT209), the BamHI and SalI fragment from pDualLuc (Deniz et al., RNA 15:932-46 (2009)) containing the PGK1 promoter, Renilla luciferase ORF, CrPV IGR IRES (nucleotides 6028-6213), and the ΔATG firefly luciferase was subcloned into the BamHI and SalI sites of the pRS425 plasmid (Christianson et al., Gene 110:119-22 (1992)). The IGRmut negative control pSRT210 was generated by site-directed mutagenesis using specific primers (ΔPKI_sense, 5′-CAGATTAGGTAGTCGAAAAACCTAAGAAATTT AGGTGCTACATTTCAAGATT-3′ (SEQ ID NO:3); ΔPKI_antisense, 5′-AATCTTGAA ATGTAGCACCTAAATTTCTTAGGTTTTTCGACTACCTAATCTG-3′ (SEQ ID NO:4) (Deniz et al., RNA 15:932-46 (2009)). The pΔEMCV plasmid (Carter and Sarnow, J. Biol. Chem. 275:28301-7 (2000)) was modified to facilitate cloning by changing the Apa1 restriction site downstream from the firefly luciferase cistron to BamHI, generating pSRT222. To construct the mammalian dicistronic IGR IRES reporter pSRT206, the NheI to XhoI fragment from pDualLuc (Deniz et al., RNA 15:932-46 (2009)), containing the Renilla luciferase CrPV IGR IRES (nucleotides 6028-6213) and DATG firefly luciferase was cloned into the NheI and BamHI sites of pSRT222. The readthrough and miscoding reporters were described previously (Keeling et al., RNA 10:691-703 (2004); Salas-Marco and Bedwell, J. Mol. Biol. 348:801-15 (2005)). The frame-shifting reporters were described previously (Harger and Dinman, RNA 9:1019-24 (2003)).

### Luciferase Assays

The IRES and frame-shifting luciferase assays were performed as described previously (Deniz et al., RNA 15:932-46 (2009)). Briefly, the yeast strains were transformed with the indicated reporter plasmid. To measure luciferase activity, cells were grown in SD media at 30° C. to mid-log phase. One OD₆₀₀ of cells was pelleted and lysed with 100 mL of 13 passive lysis buffer (PLB) for 2 minutes. Luminescence for each strain was measured using the Dual Luciferase assay kit (Promega; Madison, Wis.), following the manufacturer's protocol, with a Lumat LB 9507 luminometer (Berthold; Oak Ridge, Tenn.). Each assay was performed in triplicate. IRES activity is expressed as the firefly/Renilla luciferase ratio, normalized to the firefly/Renilla luciferase ratio of the wild-type strain.

Frame-shifting activity was measured using dual luciferase frame-shifting reporters (Harger and Dinman, RNA 9:1019-24 (2003)). Frame-shifting is expressed as the firefly/Renilla luciferase ratio of the frame-shifting reporter divided by the firefly/Renilla luciferase ratio of the control, which lacks a frame-shifting signal and has both luciferases in the same reading frame. Readthrough and miscoding was measure using reporters. Briefly, 1×10⁴ cells were harvested in mid-log phase, and dual luciferase assays were performed in quadruplicate according to manufacturer's protocols (Promega; Madison, Wis.). Firefly luciferase was translated when a readthrough or miscoding event occurred at the stop codon following the Renilla luciferase ORF. The amount of firefly luciferase activity was normalized to Renilla luciferase activity as an internal control. This value was then divided by the firefly luciferase activity normalized to Renilla luciferase from a reporter with no stop or a sense codon present, which would theoretically be 100% readthrough or miscoding, thus giving us a percent readthrough or miscoding value for each reporter. Thus, the percent readthrough or miscoding for each strain is expressed as the firefly/Renilla luciferase activity ratio (stop codon or miscoding reporter) divided by the firefly/Renilla luciferase activity ratio (sense codon or miscoding reporter) multiplied by 100.

To measure luciferase activities in HeLa cells, cells from a six-well plate were washed with phosphate-buffered saline (137 mM NaCl, 2.7 mM KCl, 10 mM sodium phosphate dibasic, 2 mM potassium phosphate at pH 7.4) and transferred to a microcentrifuge tube. Cells were pelleted by centrifugation and lysed for 15 minutes at room temperature with 200 mL of 13 PLB (Promega), and 20 mL of lysate were assayed using a Lumat LB 9507 luminometer (Berthold) according to the manufacturer's protocol (Promega). All assays were performed in triplicate.

### Polysome Profiles

Yeast strains were grown in synthetic minimal media to mid-log phase (OD₆₀₀=0.6). Cells were chilled on ice and cyclohexamide was added to a final concentration of 0.1 mg/mL. Cells were harvested by centrifugation (13,000 g, 5 minutes at 4° C.) and washed once with lysis buffer (20 mM Tris-HCl at pH 8.0, 140 mM KCl, 1.5 mM MgCl2, 0.5 mM DTT, 1% Triton X-100, 0.1 mg/mL cyclohexamide, 1 mg/mL heparin). After centrifugation (2000 g, 5 minutes, 4° C.), pellets were resuspended in lysis buffer and cells were lysed by glass bead beating. Lysates were cleared by centrifugation and layered on top of a 20%-50% sucrose gradient made in gradient buffer (20 mMTris-HCl at pH 8.0, 140 mM KCl, 5 mM MgCl2, 0.5 mM DTT, 0.1 mg/mL cyclohexamide, 1 mg/mL heparin). Gradients were processed by centrifugation in a Beckman SW41 rotor at 151,263 g for 160 minutes at 4° C. Fractions were collected, and the A₂₅₄ was recorded using an ISCO UA-5 absorbance monitor (Teledyne; Thousand Oaks, Calif.).

### 40S-Binding Assays

Yeast were grown in YPD (wild type or rps25aΔbΔ) or synthetic minimal media (rps25aΔbΔ+pS25A) to an OD₆₀₀ of 1.0. Then, cells were harvested and lysed by glass bead beating in ribo lysis buffer (20 mM HEPES at pH 7.4, 100 mM KOAc at pH 7.6,
2.5 mM Mg(OAc)₂, 1 mg/mL heparin, 2 mM DTT, Complete protease inhibitor tablets EDTA-free (Roche)). Cell lysates were clarified by centrifugation, layered over a sucrose cushion, and spun in a Beckman Type 42.1 rotor at 123,379 g for 237 minutes to pellet the polysomes. The polysomes were resuspended in a high-salt wash (20 mM HEPES at pH 7.4, 100 mM KOAc at pH 7.6, 2.5 mM Mg(OAc)₂, 500 mM KCl, 1 mg/mL heparin,
2 mM DTT] for 1 h, layered over a sucrose cushion [20 mM HEPES at pH 7.4, 100 mM KOAc at pH 7.6, 2.5 mM Mg(OAc)2, 500 mM KCl, 1 M sucrose, 2 mM DTT], and centrifuged in a Beckman TLA 100.3 rotor at 424,480 g for 30 minutes. Polysomes were released from the mRNA by the addition of puromycin (4 mM), and the ribosomal subunits were separated by centrifugation through a 5%-20% sucrose gradient (50 mM HEPES at pH 7.4, 500 mM KCl, 5 mM MgCl₂, 0.1 mM EDTA, 2 mM DTT). The gradients were fractionated, fractions containing the 40S subunits were concentrated in a Microcon centrifugal concentrator (Millipore; Billerica, Mass.), and the gradient buffer was exchanged for subunit storage buffer (20 mM Hepes•KOH at pH 7.4, 100 mM KOAc at pH 7.6, 2.5 mM Mg(OAc)₂, 250 mM sucrose, 2 mM DTT). To evaluate the integrity of the purified subunits, RNA was extracted from 20 pmol of purified 40S subunits in ribosome extraction buffer (0.3 M NaOAc at pH 5.0, 12.5 mM EDTA, 0.5% SDS) with phenol (pH 7.0) three times, and once with chloroform. The RNA was precipitated with ethanol and 1 pmol of RNA was separated on a 5% denaturing polyacrylamide gel and visualized with methylene blue (0.04% in 0.5 M NaOAc at pH 5.0).

Radiolabeled CrPV IGR IRES RNA was transcribed from the NarI linearized monocistronic luciferase plasmid (Wilson et al., Cell 102:511-20 (2000)). Radiolabeled transcripts were generated with α-³²P-UTP using the T7 RiboMax Transcription kit (Promega). The transcripts were gel-purified on a 6% denaturing polyacrylamide gel and eluted for 12 hours in elution buffer (0.5 M NH₄OAc, 1 mM EDTA, 0.1% SDS). The RNA was extracted once with acid phenol:chloroform (3:1) (Ambion; Austin, Tex.), precipitated with ethanol, and resuspended in H2O.

For the native gel shifts 1 nM radiolabeled RNA with 0-286 nM 40S subunits in 1× recon buffer (30 mM HEPES KOH at pH 7.4, 100 mM KOAc at pH 7.6, 5 mM MgCl₂, 2 mM DTT) was incubated for 15 minutes at room temperature. Complexes were separated on a 4% nondenaturing polyacrylamide gel. The bands were visualized using a PhosphorImager (Molecular Dynamics Inc., Sunnyvale, Calif.). Filter binding assays were performed with 100 nM purified 40S subunits at a range of concentrations of radiolabeled IRES RNA (from 2 nM to 300 nM) in 1× recon buffer with 50 ng/μL noncompetitor RNA transcribed from the pCDNA3 vector linearized with EcoRI. Reactions were incubated for 20 minutes at room temperature, followed by filtration through Whatman Protran nitrocellulose filters (Sigma; St. Louis, Mo.). The filters were washed twice with 1 mL of 13 recon buffer and counted in scintillation fluid using aWallac 1409 scintillation counter (Perkin Elmer; Waltham, Mass.). K_{d} values were calculated from three independent experiments.

rRNA Processing

To examine rRNA processing, yeast strains were transformed with pRS426 (Christianson et al., Gene 110:119-22 (1992)), a 2μ vector with a URA3 backbone, and were grown in selective media lacking uracil to 0.8 OD₆₀₀. One-hundred microliters of [5,6-³H] uracil (50 Ci/mmol, Perkin-Elmer) were added to the culture for a final concentration of 0.100 mCi for 3 minutes at 30° C., and the [5,6-³H] uracil was chased with 0.064 mg/mL cold uracil. Samples were removed at 0, 2, 5, and 15 minutes after addition of the cold uracil and were flash-frozen in liquid nitrogen. RNA was isolated from the samples and run on a denaturing 1% agarose gel in MOPS Buffer (20 mM MOPS, 5 mM NaOAc, 1 mM EDTA at pH7.0), 1% agarose, and 16% formaldehyde. RNA was transferred to a HyBond-N⁺ nylon membrane (GE Healthcare; Piscataway, N.J.), soaked in amplify (GE Healthcare), dried, and visualized using autoradiography.

### Protein Synthesis Rate

Protein synthesis rates were determined by [³⁵S] methionine incorporation. Briefly, wild-type and rps25aΔbΔ yeast strains were grown in selective media without methionine to an OD₆₀₀ 0.5. At the initial time point, each culture was adjusted with cold methionine (50 mM) and [³⁵S] methionine (1 mCi/mL; EasyTag EXPRESS³⁵S, 74MBq, Perkin Elmer). At 15 minute intervals, the OD₆₀₀ was determined, and 1 mL of culture was added to 200 mL of cold 50% trichloroacetic acid (TCA). The samples were incubated for 10 minutes on ice and 20 minutes at 70° C., and were filtered through a Whatman GF/A filter. The filters were washed with 10 mL of 5% cold TCA, followed by 10 mL of 95% ethanol, and were dried for 10 minutes prior to scintillation counting. The protein synthesis rates were determined from three independent experiments.

siRNA and DNA Transfections

Custom double-stranded siRNAs that target Rps25 were purchased from Ambion: sense, 5′-GGACUUAUCAAACUGGUUUtt-3′ (SEQ ID NO:5), and antisense, 5′-AAACCAGUUUGAUAAGUCCtt-3′ (SEQ ID NO:6) (siRNA ID #142220). The negative control, a nontargeting siRNA, was purchased from Dharmacon (siCONTROL Nontargeting siRNA #1) (Dharmacon; Lafayette, Colo.). HeLa cells were transfected with siRNA by combining 75 mM siRNA with 5 mL of siPORT NeoFX transfection reagent (Ambion) in a 20-mm plate, which was overlaid with 2 3 105 HeLa cells in antibiotic-free Complete media. DNA transfections were performed 24 or 48 h post-siRNA treatment using Lipofectamine 2000 (Invitrogen; Carlsbad, Calif.) according to the manufacturer's protocol, using 4 mg of DNA per well. Cells were harvested for either luciferase analysis at 72 or 96 hours or Northern analysis.

shRNA lentiviral vectors were constructed using the pLVTHM vector (Addgene plasmid 12247; Addgene; Cambridge, Mass.). The rpS25 shRNA oligos (sense, 5′-cgcgtccccGGACTTATCAAACTGGTTTttcaagagaAAACCAGTTTGATAAGTCCttttt ggaaat-3′ (SEQ ID NO: 12) and antisense, 5′-cgatttccaaaaaCCTGAATAGTTTGACCAAA agagaacttTTTGGTCAAACTATTCAGGcccct-3′ (SEQ ID NO:13)) were commercially synthesized (IDT DNA Technologies; Coralville, Iowa), phosphorylated, (T4 Kinase, Promega) and annealed before ligating into the ClaI/MluI restricted pLVTHM vector. Cloning was verified by sequencing. Virus was generated by cotransfection of the lentiviral vector, packaging plasmid (psPAX2 [addgene plasmid 12260]) and a VSV-G envelop plasmid (pMG2.G [addgene plasmid 12259]) into HEK293T cells. After 24 hours, supernatant was collected every 12 hours for 2 days. The viral supernatant was filter sterilized using a 0.2 um filter and used directly on the target cell line.

### Northern Analysis

Total RNA was harvested from siRNA-treated cells 48, 72, and 96 hours post-transfection with TRIzol (Invitrogen Life Technologies; Carlsbad, Calif.) according to the manufacturer's directions. Four micrograms of RNA were separated on a denaturing agarose gel (0.8% agarose, 16% formaldehyde) in MOPS buffer and transferred to Zeta-Probe membrane (Bio-Rad; Hercules, Calif.). A radiolabeled Rps25 probe was generated with the Prime-a-Gene kit (Promega) and ³²P-dCTP (PerkinElmer) using a PCR product amplified from a HeLa cDNA pool with the following primers: sense, 5′-ATGCCGCCTA AGGACGAC-3′ (SEQ ID NO:7), and antisense, 5′-TCATGCATCTTCACCAGC-3′ (SEQ ID NO:8). The membrane was hybridized according to the manufacturer's protocol and analyzed by autoradiography. The membranes were stripped at 95° C. in stripping buffer (0.1% SSC, 0.5% SDS) and reprobed for β-actin (primers: sense, 5′-GCACTCT TCCAGCCTTCC-3′ (SEQ ID NO:9), and antisense, 5′-GCGCTCAGGAGGGAGCA AT-3′ (SEQ ID NO:10)).

### Example 1

### Rps25 is Essential for IRES Activity

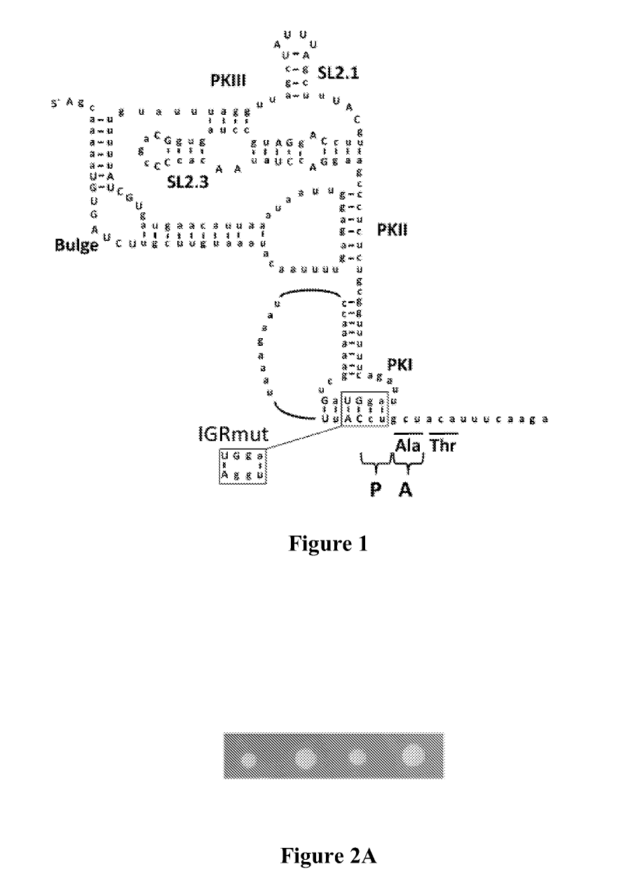

The cricket paralysis virus (CrPV) IGR IRES is ˜180 nucleotides long, and in vitro it is able to bind directly to the 40S subunits followed by the recruitment of the 60S subunit to assemble translationally competent 80S ribosomes. It is able to initiate translation in vivo in both yeast and mammalian cells. Thus, it serves as a good model for IGR IRES interactions with the ribosome. The IGR IRES (FIG. 1) consists of three pseudoknot structures (PKI, PKII, and PKIII). Areas with the highest sequence conservation across the Dicistroviridae family (FIG. 1, see capitalized nucleotides) are located int eh loop regions and have been predicted to interact directly with the ribosome. Stem-loop 2.1 (SL 2.1), SL 2.3, and PKIII are believed to be responsible for 40S subunit recruitment based on mutational analysis of the stem-loops, which leads to a reduction in translation and 40S complex formation (Jan and Sarnow, J. Mol. Bio. 324:889-902 (2002); Costantino and Kieft, RNA 11:332-43 (2005)). Crystallization and cryo-electron microscopy (cryo-EM) studies of the IGR IRES revealed that the IRES forms a tightly packed core from which SL 2.1 and SL 2.3 protrude adjacent to one another to contact the 40S ribosome (Spahn et al., Cell 118:465-75 (2004); Pfingsten et al., Science 314:1450-4 (2006); Schuler et al., Nat. Struct. Mol. Biol. 13:1092-6 (2006); Costantino et al., Nat. Struct. Mol. Biol. 15:57-64 (2008)). PKII and the bulge region are predicted to interact with the 60S subunit (Schuler et al., Nat. Struct. Mol. Biol. 13:1092-6 (2006)). PKI is positioned in the P site of the ribosome to initiate translation at the adjacent codon positioned in the A site (Wilson et al., Cell 102:511-20 (2000)).

Two lines of evidence suggest that Rps25 may interact with the IGR IRES. First, Rps25 cross-links to the Plautia stali intestine virus (PSIV) IGR IRES in vitro (Nishiyama et al., Nucleic Acids Res. 35:1514-21 (2007)). Second, the cryo-EM model of the CrPV IGR IRES bound to 80S ribosomes predicts that SL2.1 interacts with Rps5 and SL2.3 interacts with an adjacent protein density that has no prokaryotic homolog (Schuler et al., Nat. Struct. Mol. Biol. 13:1092-6 (2006)). Cross-linking experiments with eukaryotic ribosomes identified Rps25 as being in close proximity to Rps5 (Uchiumi et al., J. Biochem. 90:185-93 1981).

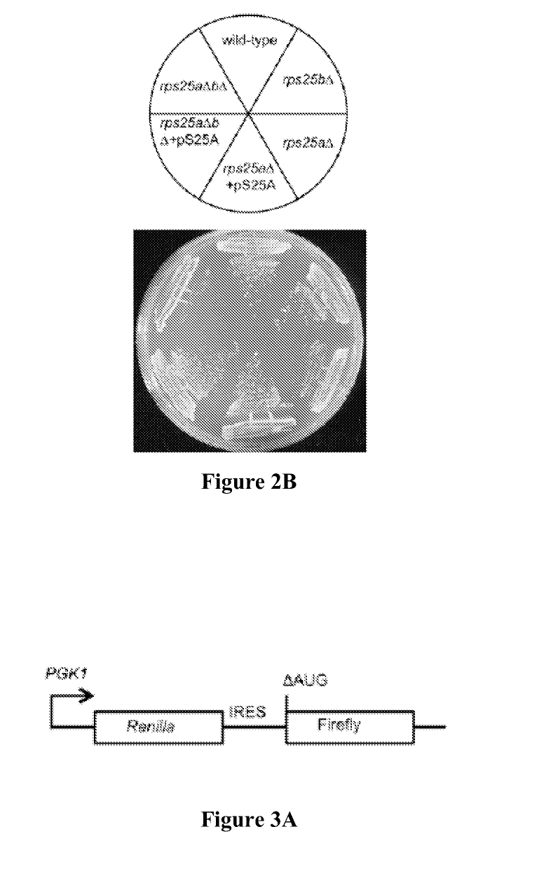

To determine whether Rps25p could be involved in CrPV IGR IRES activity in vivo, a yeast knockout strain for RPS25 was generated. Similar to most ribosomal proteins in Saccharomyes cerevisiae, RPS25 is duplicated in the genome. The genes encode proteins Rps25a and Rps25b, which differ only by one amino acid at the C-terminal end. rps25aΔ and rps25bΔ haploids were mated to obtain diploids. Sporulation of the diploids and dissection of the tetrads consistently resulted in two colonies that grew at wild-type growth rates and two colonies that grew more slowly (FIG. 2). RPS25A and RPS25B deletions were confirmed by both PCR and Southern analysis, demonstrating that, in agreement with previous studies, Rps25p is not an essential protein in S. cerevisiae (Ferreira-Cerca et al., Mol. Cell 20:263-75 (2005)). RPS25A accounts for ˜66% of the Rps25p in the cell (Ghaemmaghami et al., Nature 425:737-41 (2003)), which may explain why deletion of RPS25B did not result in any defect in cell growth. A plasmid expressing RPS25A was able to rescue the growth defects of rps25aΔ and rps25aΔbΔ strains (FIG. 2B).

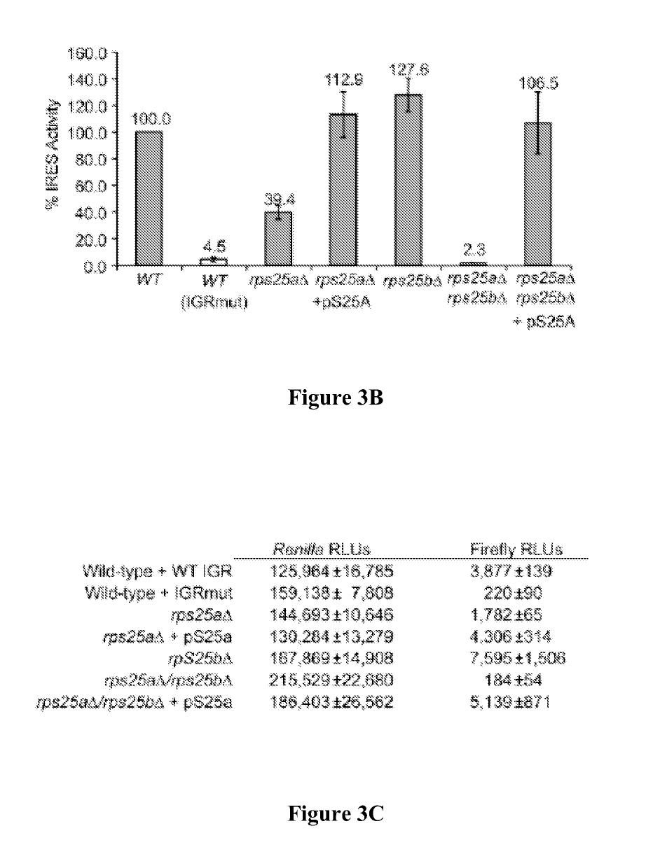

To determine if Rps25 is required for IRES-mediated translation in vivo, a dicistronic reporter containing the CrPV IGR IRES inserted between Renilla and firefly
luciferase ORFs was transformed into wild-type and mutant yeast strains (FIG. 3A). Since the CrPV IGR IRES initiates at an alanine codon rather than an AUG methionine codon, the AUG start codon of the firefly luciferase ORF was deleted to eliminate expression of active firefly luciferase from transcripts generated by cryptic promoters (Deniz et al., RNA 15:932-46 (2009)). Firefly luciferase activity is sensitive to N-terminal truncations, such that deletion of amino acid residues 3-10 decreases firefly luciferase activity to 0.1% of wild-type levels (Sung and Kang, Photochem. Photobiol. 68:749-53 (1998)). Therefore, by deleting the initiating AUG codon, any transcripts generated from cryptic promoters that could use a cap-dependent mechanism to initiate translation will result in no firefly activity, since the next inframe AUG codon is 29 codons downstream. Furthermore, the CrPV IGR IRES is active in wild-type yeast strains, while the inactive IGRmut that disrupts the basepairing in PKI does not have any IRES activity (FIG. 3B; Deniz et al., RNA 15:932-46 (2009)). It was found that the rps25bΔ strain has similar IGR IRES activity to the wild type. In contrast, the rps25aΔ strain exhibits ˜40% IRES activity, while the rps25aΔbΔ mutant strain has virtually no IRES activity, at 2.3% of wild type. When RPS25A is expressed from a plasmid, IRES activity is restored to wild-type levels for both the rps25aΔ and rps25aΔbΔ strains (FIGS. 3B and 3C). In contrast, cap-dependent translation is not affected by the lack of Rps25 (FIG. 3C, Renilla RLUs). Taken together, these results demonstrate that the IGR IRES activity but not cap-dependent translation is dependent on the Rps25 protein.

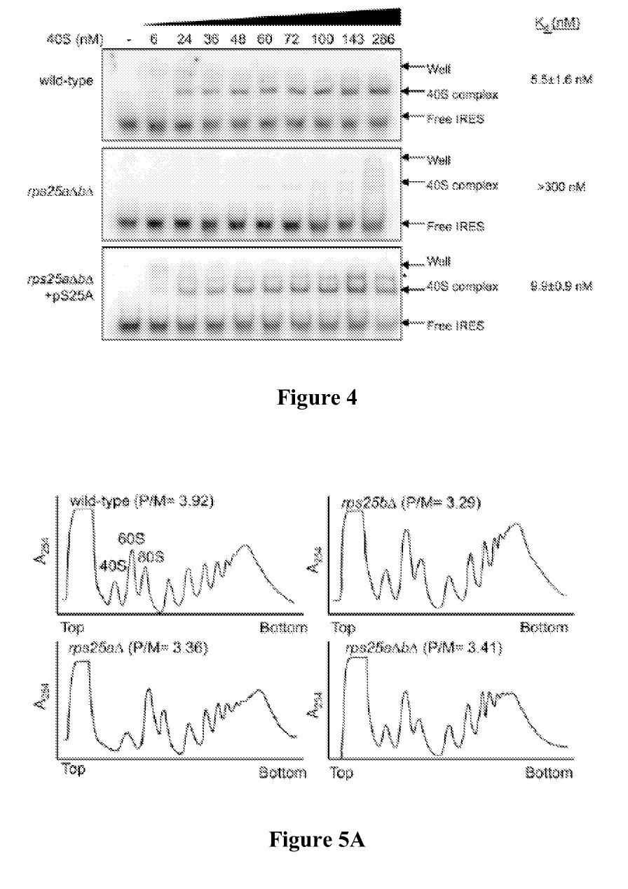

The lack of IGR IRES activity in the rps25aΔbΔ strain could be caused by either a failure of the IRES to recruit the 40S subunit, or a failure in some other downstream process, such as 60S subunit joining or pseudotranslocation. The IGR IRES has been shown to bind to purified 40S subunits, followed by recruitment of the 60S subunit
to form 80S complexes in vitro (Wilson et al., Cell 102:511-20 (2000); Jan et al., Proc. Natl. Acad. Sci. USA 100:15410-5 (2003); Pestova and Hellen, Genes Dev. 17:181-6 (2003)). To determine if the decrease in IRES activity was due to an inability of the IRES to bind 40S subunits, native gel shifts were performed with radiolabeled CrPV IGR IRES RNA and purified 40S ribosomal subunits from either wild-type, rps25aΔbΔ, or rps25aΔbΔ+pS25A yeast strains. The IGR IRES RNA was able to bind to wild-type 40S subunits with a dissociation constant of 5.5 nM, which is also evidenced by the shift in mobility of the radiolabeled RNA (FIG. 4, top). However, when Rps25p was absent, the ability of the IGR IRES RNA to bind the 40S subunits was severely impaired even at the highest concentrations of 40S subunits (FIG. 4, middle). When Rps25 is expressed from a plasmid, binding of the IGR IRES to 40S subunits is restored (FIG. 4, bottom). In the rps25aΔbΔ+pS25A gel shift, the formation of 80S complexes was observed (FIG. 4, asterisk) due to some contaminating 60S subunits in the 40S preparation. A gel of the ribosomal RNA (rRNA) isolated from the purified subunits demonstrated that the rRNA is intact, indicating the lack of 40S subunit binding by the rps25aΔbΔ ribosomes is due to the absence of Rps25 protein and not the degradation of the subunits. These binding assays are consistent with the IGR IRES activity determined in vivo, where the rps25aΔbΔ yeast resulted in no IRES activity (FIG. 3). Both IRES activity and 40S ribosomal subunit binding were rescued to wild-type levels when Rps25 was expressed from a plasmid. Thus, deletion of Rps25 in S. cerevisiae essentially eliminates IGR IRES activity in vivo due to the inability of the IRES to recruit 40S subunits.

### Example 2

### Rps25 Deletion has Only Slight Effects on Global Translation and Ribosome Fidelity

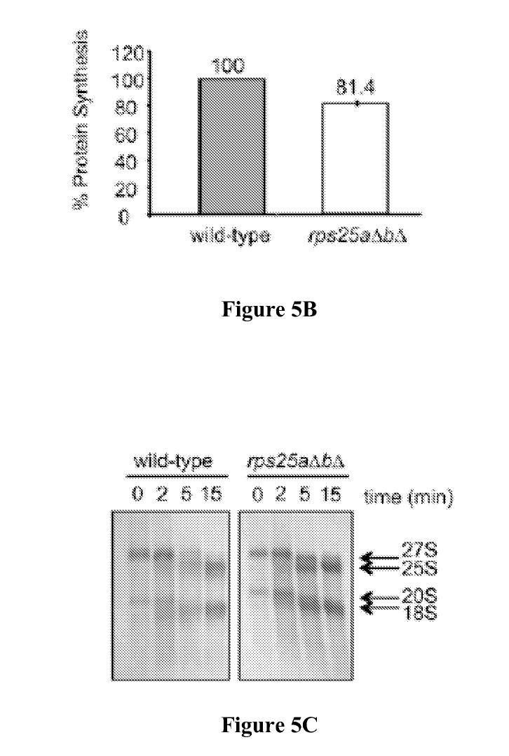

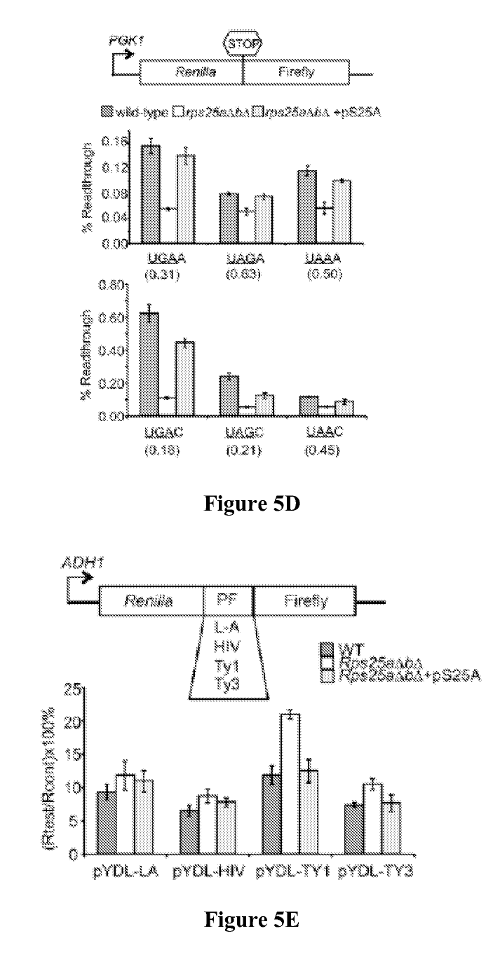

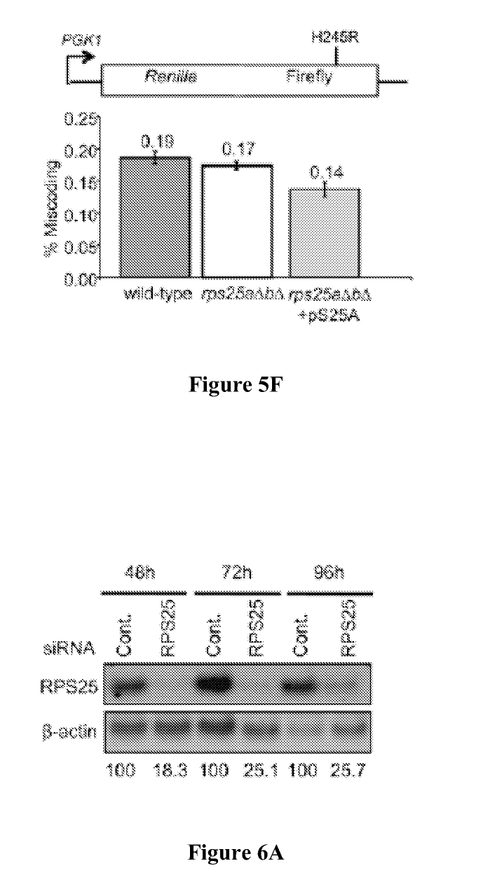

Since knockout of the RPS25 genes results in a dramatic decrease in IRES-mediated translation, it was sought to determine whether Rps25 was required for any other ribosomal functions. A polysome analysis on wild-type, rps25aΔ, rps25bΔ, and rps25aΔbΔ yeast was performed (FIG. 5A). All of the deletion strains had a similar polysome profile and polysome to monosome ratio. Since no decrease in the polysome fractions was observed, it was determined that deletion of one or both copies of Rps25 does not cause a significant defect in global translation initiation. This is consistent with what has been shown previously (Ferreira-Cerca et al., Mol. Cell 20:263-75 (2005)). These results are also consistent with the observation that the Renilla luciferase activity was similar to wild-type activity in all of the deletion strains. To more carefully evaluate the effects of Rps25 deletion on global protein synthesis, ³⁵S-methinione incorporation assays were performed. These results indicate that the rps25aΔbΔ strains exhibit a slight decrease (19%) (FIG. 5B) in global protein synthesis relative to the wild-type strain. The amounts of 40S and 60S subunits appear to be similar in all strains, suggesting no defect in ribosome biogenesis. To more carefully evaluate this, pulse-chase experiments on wild-type and rps25aΔbΔ strains were performed (FIG. 5C). The appearance of the fully processed 25S and 18S rRNA species are slightly delayed in the double-deletion mutant. However, there is no apparent accumulation of pre-rRNA species, and the amounts of 25S and 18S rRNA appear to be similar between the wild-type and rps25aΔbΔ strains. The slight decrease observed in the protein synthesis rate or the delayed rRNA biogenesis rate could contribute to the observed slow-growth phenotype.

To determine whether ribosomes lacking Rps25p exhibited an increase in translational errors, readthrough of stop codons, miscoding, and programmed ribosomal frameshifting (PRF) was examined. The efficiency of stop codon recognition using dual luciferase readthrough reporters was measured (FIG. 5D, top). Translation termination is dependent not only on the stop codon, but also on the surrounding context, in particular, the nucleotide directly following the stop codon (tetranucleotide termination signal) (Bonetti et al., J. Mol. Biol. 251:334-45 (1995)). The percent readthrough in wild-type, rps25aΔbΔ, and rps25aΔbΔ with pS25A strains using dual luciferase readthrough reporters with either an adenosine or a cytosine as the following nucleotide for each of the three stop codons was assayed. It was observed that the rps25aΔbΔ strain exhibited an increase in stop codon recognition as compared with the wild-type strain for all of the tetranucleotide stop codons tested (FIG. 5D). Importantly, the pS25A rescue plasmid returned readthrough to the wild-type levels. The consistent decrease observed in readthrough demonstrates that this is a general phenomenon that is not specific to any particular stop codon.

In addition to readthrough, the effect of RPS25 deletion on PRF was also examined. Frameshifting can occur when specific signals in the mRNA induce the ribosome to change reading frames in the 3′ direction (+1 PRF) or in the 5′ direction (−1 PRF) (Namy et al., Mol. Cell 13:157-168 (2004); Brierley and Dos Ramos, Virus Res. 119:29-42 (2006); Giedroc and Cornish, Virus Res. 139:193-208 (2009)). Frameshifting is triggered by two elements: a slippery sequence where tRNA movement or misalignment is favored, and a stimulator element that enhances the process by causing a ribosomal pause. To determine if deletion of RPS25 has any affect on programmed ribosomal frameshifting, dual luciferase reporters that contain one of four viral PRF signals (L-A, HIV, Ty1, and Ty3) inserted into the region between Renilla and firefly luciferase ORFs were used (FIG. 5E, top; Harger and Dinman, RNA 9:1019-24 (2003)). L-A and HIV are both programmed −1 ribosomal frameshift signals, and the data show no difference between wild-type and rps25aΔbΔ ribosomal frameshift values (FIG. 5E). However, there is an increase in frameshifting in the rps25aΔbΔ strain for the Ty1+1 PRF signal and a slight increase for the Ty3+1 PRF signal (FIG. 5E). Ty1+1 frameshifting occurs at a 7-nt sequence in the Ty retrotransposon because of a ribosomal pause at an AGG codon in the A site of the ribosome. The availability of tRNA to decode the AGG codon is low, causing a pause and subsequent mRNA slippage. The amount of +1 frameshifting in the rps25aΔbΔ strain is still within the range of what has been reported for wild-type S. cerevisiae cells (Belcourt and Farabaugh, Cell 62:339-52 (1990)), although it is notable that this signal is nearly doubled in rps25aΔbΔ cells compared with wild type. Importantly, wild-type rates of frameshifting were restored when the pS25A rescue plasmid was present in the rps25aΔbΔ strain. Last, miscoding was examined using a dual luciferase miscoding reporter that contains a detrimental histidine-to-arginine mutation at codon 245 in the firefly luciferase (FIG. 5F, top; Salas-Marco and Bedwell, J. Mol. Biol. 348:801-815 (2005)). Misincorporation of an amino acid at this position results in an increase in firefly luciferase activity. No difference in misincorporation was observed between the wild-type and rps25aΔbΔ strains (FIG. 5F, bottom). Taken together, these results demonstrate that, in general, the ribosome is functional and deletion of Rps25p from the 40S subunit does not result in significant defects in ribosomal functions. This is in sharp contrast to its role in IGR IRES-mediated translation, where Rps25p is absolutely required for activity and binding to the 40S subunit.

### Example 3

### The Function of Rps25 in IRES-Mediated Translation is Conserved in Mammals

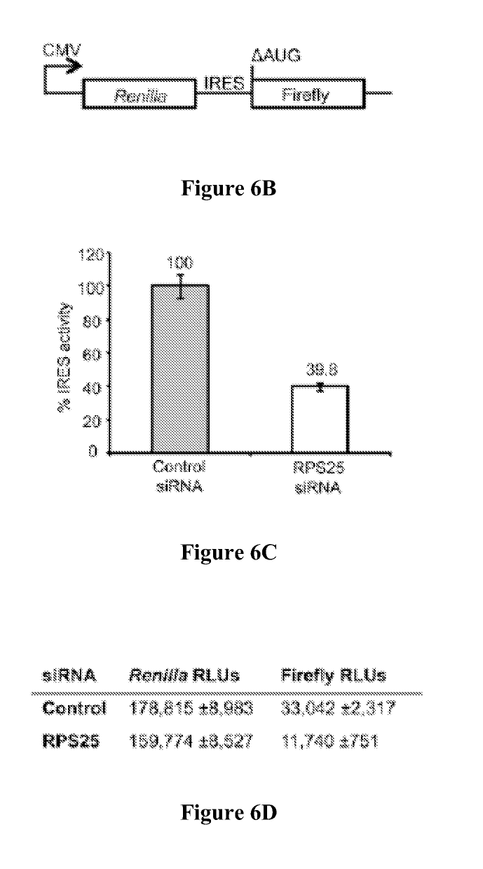

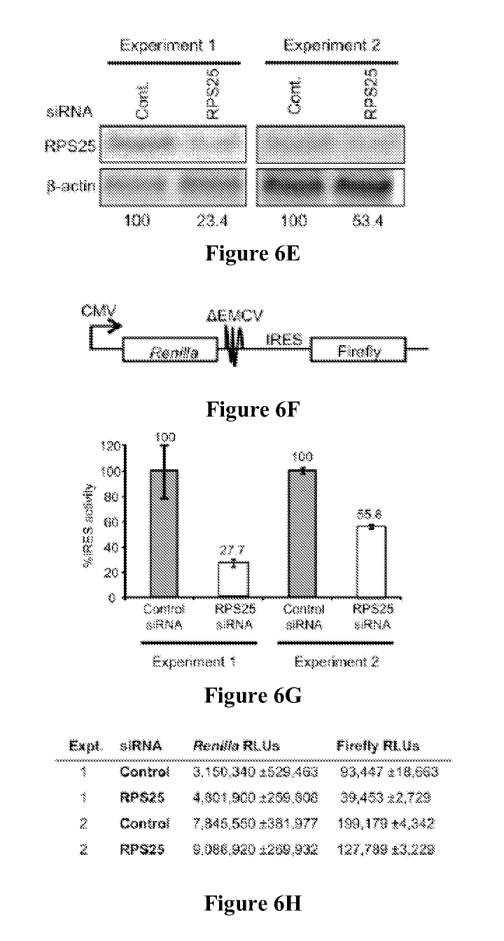

Since the IGR IRES functions to initiate translation with ribosomes from a variety of organisms, such as plants, mammals, and yeast, it was sought to determine whether the function of Rps25p in IGR IRES-mediated translation was conserved in mammalian cells. RPS25 is present in only one copy in the genome in mammals, and it is 47% identical and 71% similar to the yeast RPS25A. siRNA against the RPS25 mRNA was used to knock down expression of the Rps25 protein in HeLa cells. A 75% decrease in RPS25 mRNA was achieved (FIG. 6A). To determine whether Rps25 knockdown had any effect on IGR IRES activity in mammalian cells, a dicistronic luciferase reporter containing the CrPV IGR IRES in the intercistronic region was transfected in the cells (FIG. 6B). A 60% decrease in IGR IRES-mediated translation was observed when Rps25 was knocked down (FIG. 6C). This level of inhibition is equivalent to the inhibition observed in the rps25aΔ strain (FIG. 3), which corresponds to a 66% decrease in the Rps25 protein in the cell (Ghaemmaghami et al., Nature 425:737-41 (2003)). Also in agreement with experiments in yeast, a significant decrease in cap-dependent translation was not observed when Rps25 was knocked down (FIG. 6D). Knockdown of the Rps25 protein was unable to be confirmed due to the lack of an adequate antibody. However, since a decrease in both RPS25 mRNA and IGR IRES-mediated translation was observed, it is believed that the protein levels were also affected. It is concluded that Rps25 is required for IGR IRES function in mammalian cells.

To determine whether Rps25 is required for other IRESs, the effects of Rps25 depletion on the HCV IRES were analyzed. Either control or Rps25 siRNAs were transfected into HeLa cells to knock down Rps25 (FIG. 6E). Then, 24 hours later, the HCV IRES dicistronic reporter was transfected (FIG. 6F) and assayed for IRES activity. When Rps25 mRNA was knocked down, a dramatic decrease in HCV IRES activity was observed, demonstrating that Rps25 is also required for the HCV IRES (FIG. 6G). Further, a Rps25 shRNA was created and cloned into a lentiviral vector to knockdown Rps25.

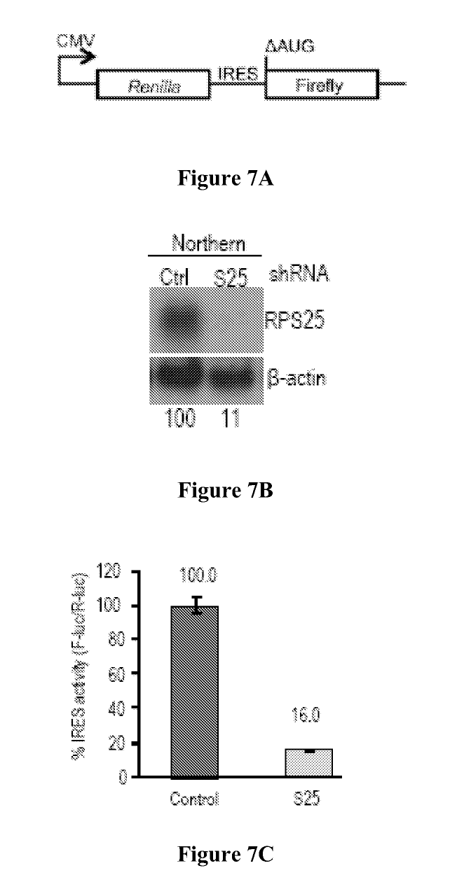

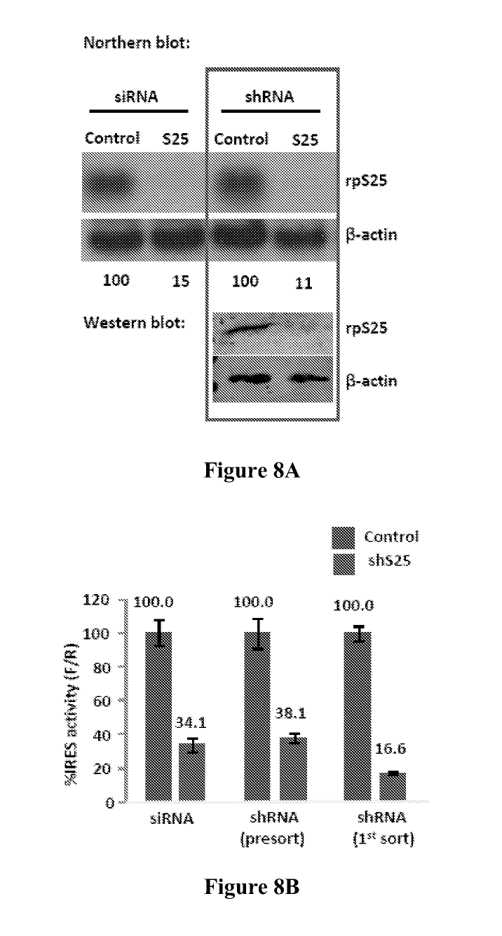

In a similar experiment, lentiviral constructs containing control or Rps25 shRNAs were transduced into HeLa cells to knock down Rps25 (FIG. 7B). Then, 24 hours later, the HCV IRES discistronic reporter (FIG. 7A) was transfected and assayed for IRES activity. When Rps25 mRNA was knocked down, a dramatic decrease in HCV IRES activity was observed (FIG. 7C), further confirming that Rps25 is required for HCV IRES activity.

### Example 4

### Rps25 is Required for IRES Mediated Translation of Other Viral and Cellular RNAs

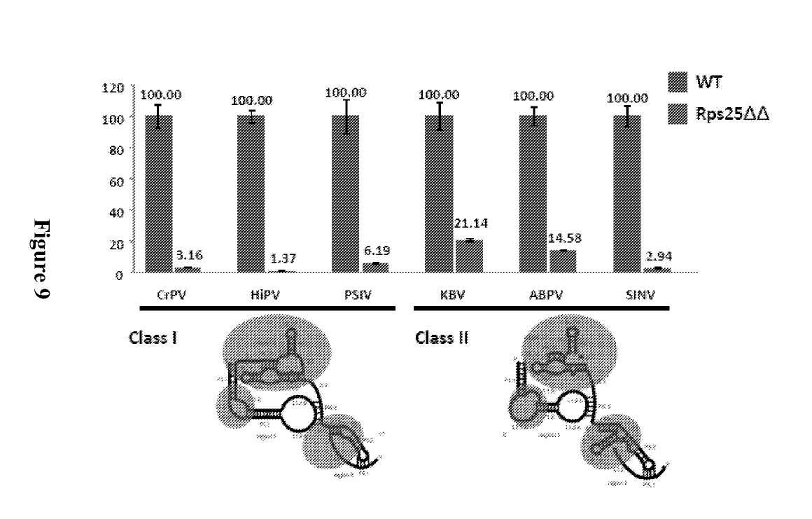

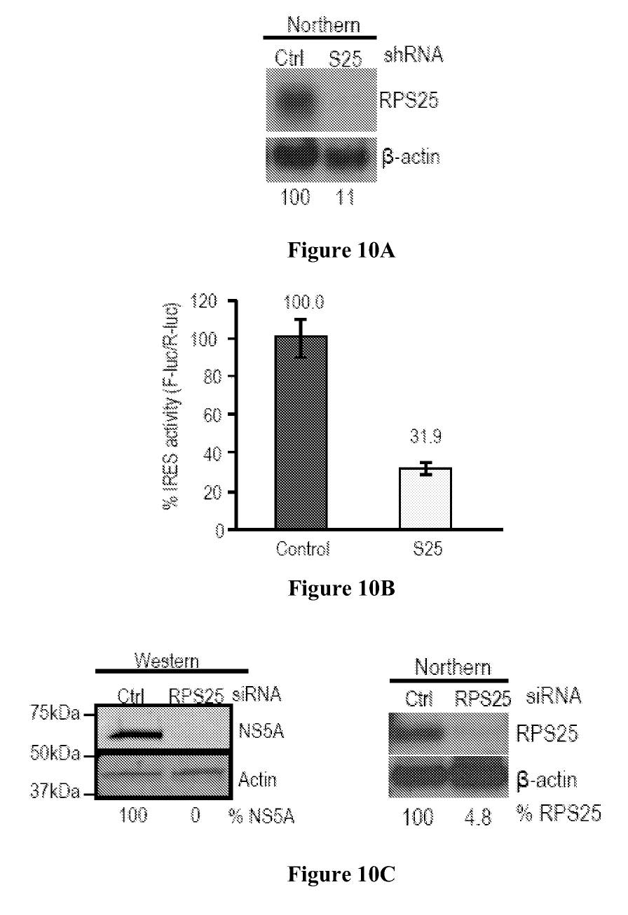

It was also determined that Rps25 was required for both classes of IGR IRESs. The CrPV belongs to the dicistroviridae family, which contains two classes of IGR IRESs. The CrPV IRES belongs to class I, whereas the class II IRESs have a larger bulge and an extra stem loop in domain III of the IRES. In experiments similar to the ones performed above, it was determined that Rps25 is essential for IRES-mediated translation of both classes of IGR IRESs (FIG. 9).

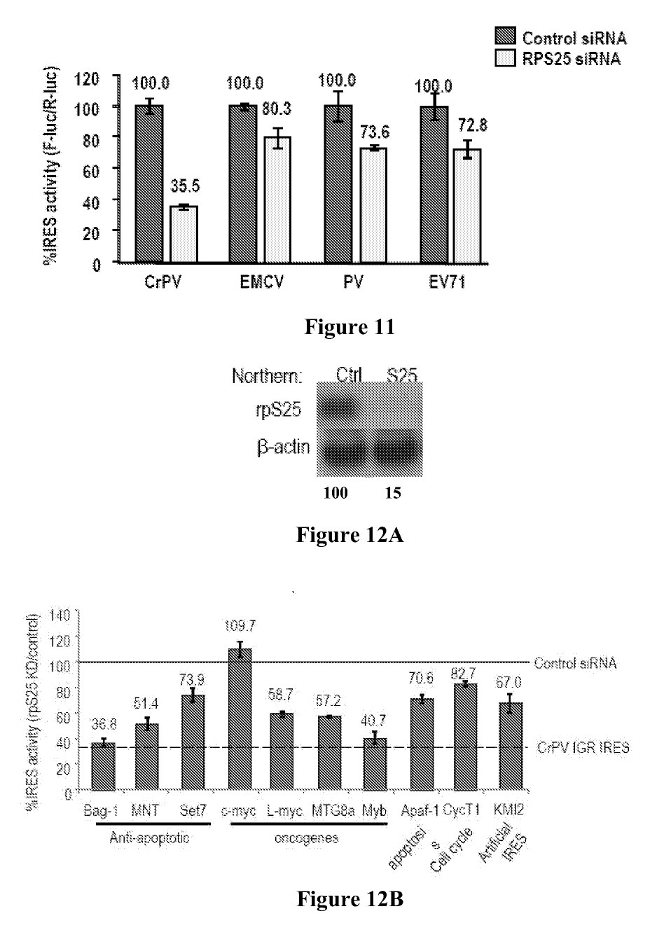

Rps25 was also shown to enhance picornaviral IRES activity. Dicistronic reporter constructs containing the Encephalomyocarditis virus IRES, the Poliovirus IRES, and the Enterovirus 71 IRES were created and used to determine the effect of Rps25 knockdown on IRES mediated translation. Knockdown of Rps25 led to reduced levels of IRES-mediated translation from these picornaviral IRES elements (FIG. 11).

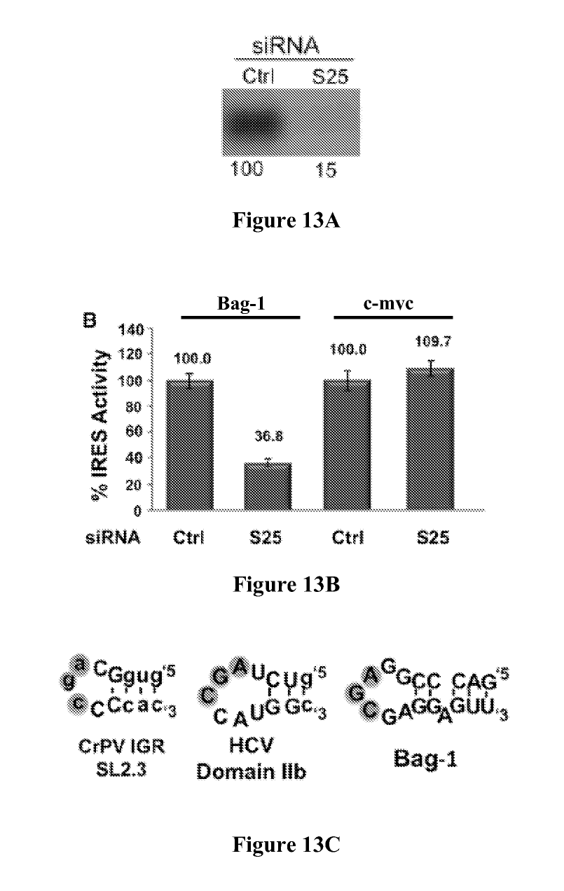

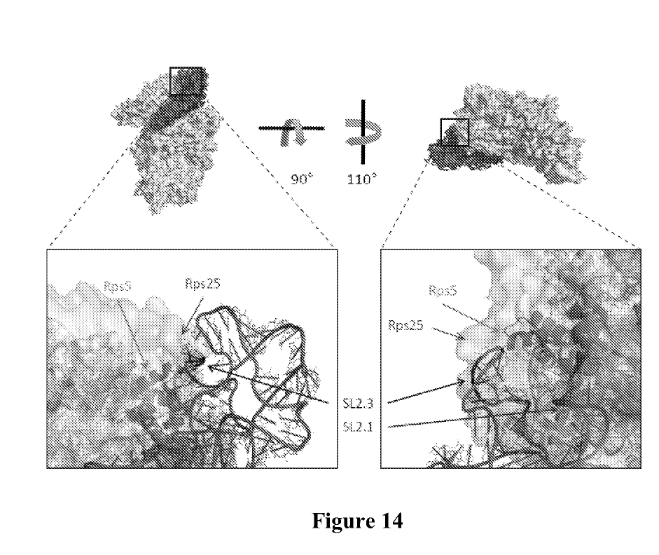

While the HCV and the CrPV IGR IRES elements are structurally and functionally different, they both share the same requirement for Rps25. To determine if Rps 25 dependency extended to ther types of IRES elements, two cellular IRESs, Bag-1 and c-myc were used to determine the if Rps25 was required for translation. It was found that the Bag-1 IRES element was dependent on Rps25 for translation (FIG. 13B). The c-myc IRES element did not depend of Rps25 for translation (FIG. 13B). Through a phylogenetic comparison of the HCV, CrPV, and Bag-1 IRESs, it was determined that a similar sequence motif was present (FIG. 13C). Specifically, they have a stem-loop that contains an AGC sequence in the loop region. Extensive site-directed mutagensis on the CrPV IGR IRES stem loop has been performed, which indicates that AGC is not the only sequence that will function, rather any sequence that has an ANY (A:adenine, N:any nucleotide, Y: pyrimidine) motif results in wild-type IRES activity or higher. This consensus sequence is consistent with all the known SL2.3 sequences in the Dicistroviridae family of viruses (Nakashima and Uchiumi, Virus Res. 139:137-47 (2009)). Interestingly, the HCV domain IIb (UAGCCAU) (SEQ ID NO:14) is 100% conserved among all HCV genotypes as well as the closely related classic swine fever virus (CSFV). Domain IIb of the HCV IRES interacts with the E site of the ribosome, which is the predicted location of Rps25 (Uchiumi et al., J. Biochem. 90:185-95 (1981); and Landry et al., Genes Dev. 23:2753-64 (2009)).

In addition to Bag-1 and c-myc IRES elements, there are also multiple cellular RNAs that are translated through the use of an IRES. Several cellular IRES elements were cloned into the dicistronic IRES reporter. Knockdown of Rps25 with siRNAs led to reduced levels of IRES-mediated translation of the cellular IRES elements (FIG. 12). It is noted that Bag-1 levels were reduced to that of the CrPV IRES element.

### Example 5

### Optimization of Transient Transfection of the HCV IRES Dual LUC Reporter into Huh7 Human Hepatocyte Cells

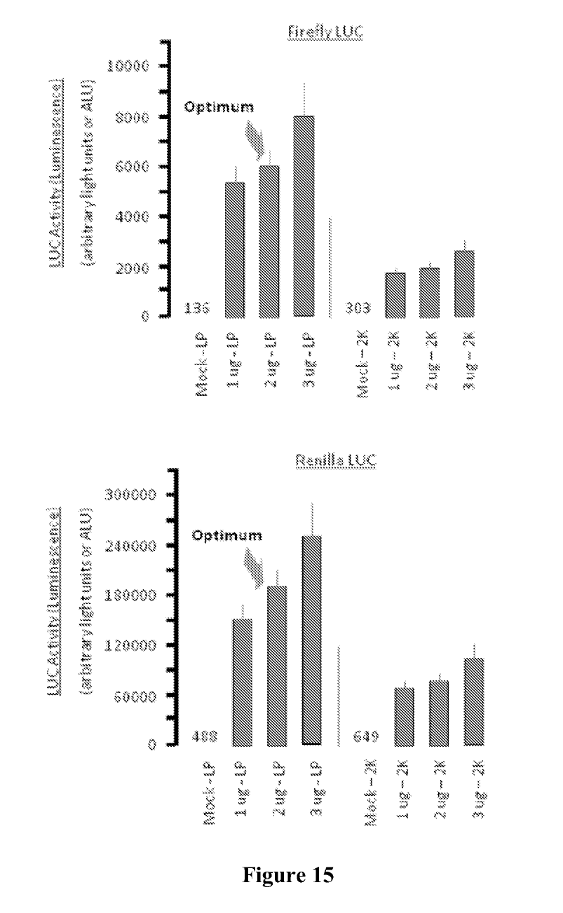

The first hurdle in the assay design and optimization was to determine if Huh7 human liver cells were easily transfectable with the use of cationic lipid transfection reagent. LipofectAMINE reagent (Gibco-BRL; Invitrogen; Carlsbad, Calif.) was not very efficacious when used alone. However, LipofectAMINE PLUS and LipofectAMINE 2000 were compared with increasing amounts of HCV IRES Dual LUC reporter plasmid (FIG. 15). LipofectAMINE 2000 was not as effective as LipofectAMINE PLUS. PLUS reagent is mixed with the plasmid DNA initially to prime the DNA for more effective transfection by LipofectAMINE due to proprietary chemistry developed by Gibco-BRL and acquired by Invitrogen. LipofectAMINE PLUS-mediated transient transfection produced an ample signal when 2 micrograms of plasmid and 6 microliters of both PLUS reagent and LipofectAMINE reagent was used per row of the 96-well plate (12 wells). This optimized condition is applied to the experimental design, optimization and implementation presented below and will be a benchmark by which all future experiments will be performed or modified (i.e., if the assay is further miniaturized to smaller wells).

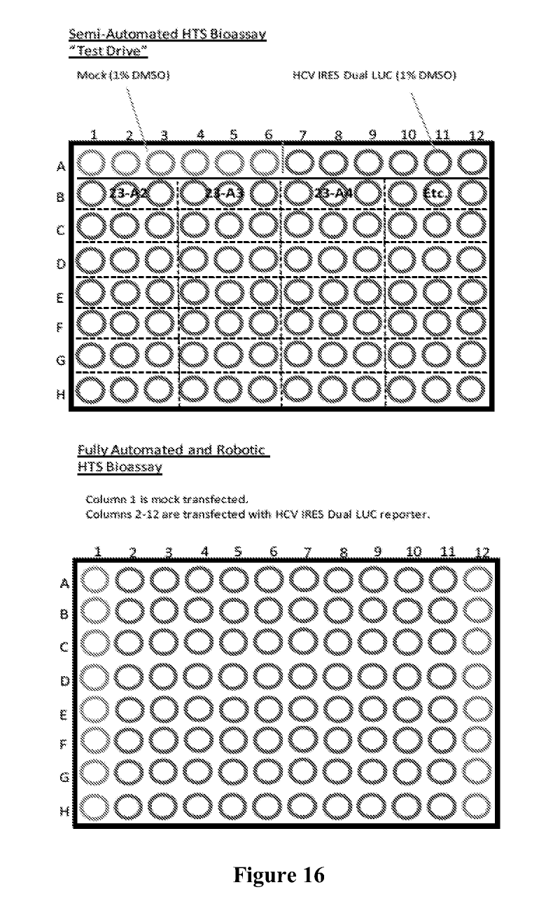

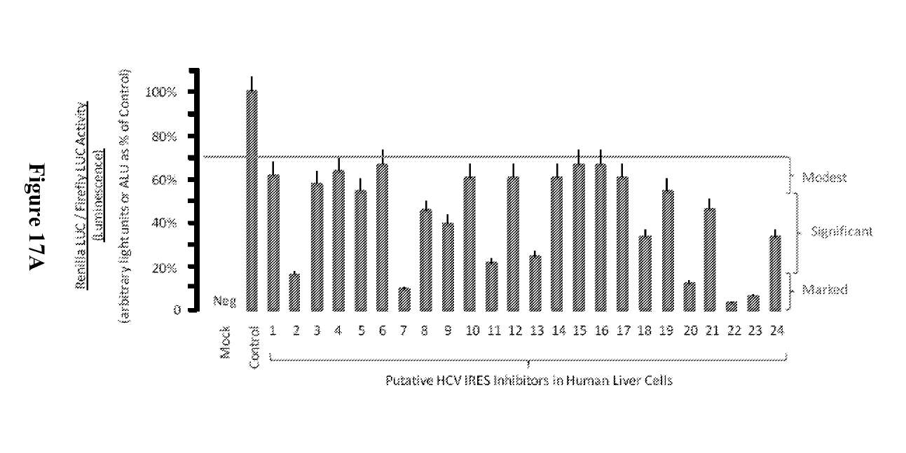

Two different 96-well microtiter plate design were used to ‘test drive’ a near optimized assay with actual test small molecules. Both designs allow each test small molecule to be screened in triplicate (i.e., in 3 different wells within the microtiter plate). For the first 160 compounds tested, the first design was used (FIG. 16, top). For the next 800 compounds to screen 960 total small molecules in the initial pilot high throughput ‘test drive,’ the second design was used (FIG. 16, bottom), which is the standard design for automated, robotic implementation of every high throughput bioassay and program. With either design, hit compounds were easily discernable because of the robust dual LUC signal achieved (see below).

### Example 6

### Small Molecule Screen Results in Identification of HCV IRES Translation Inhibitors

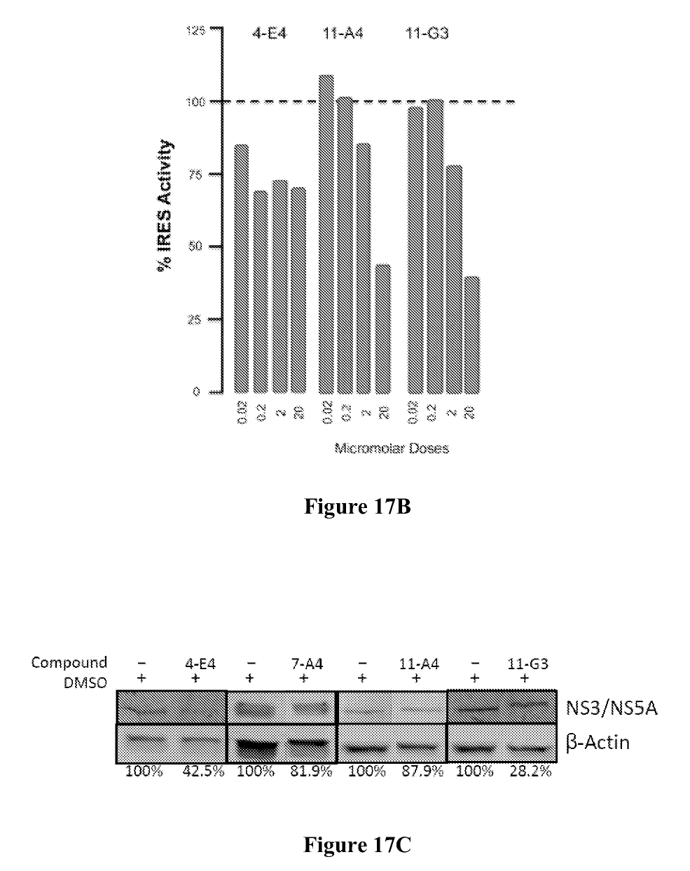

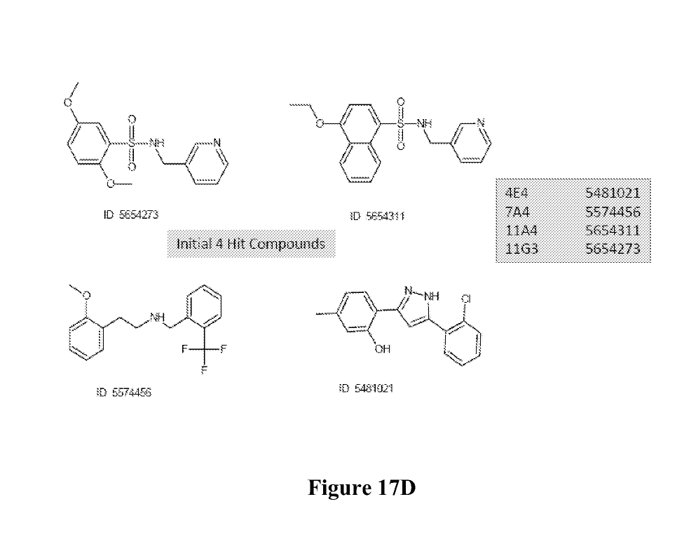

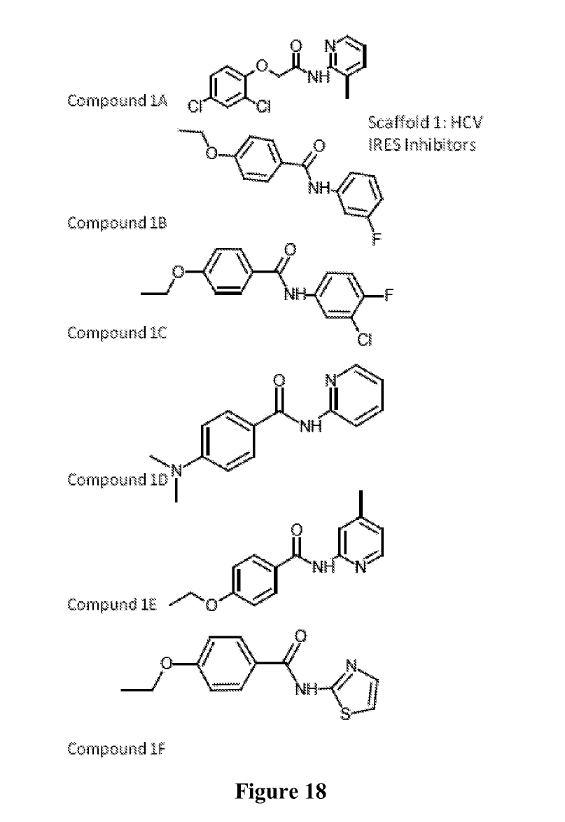

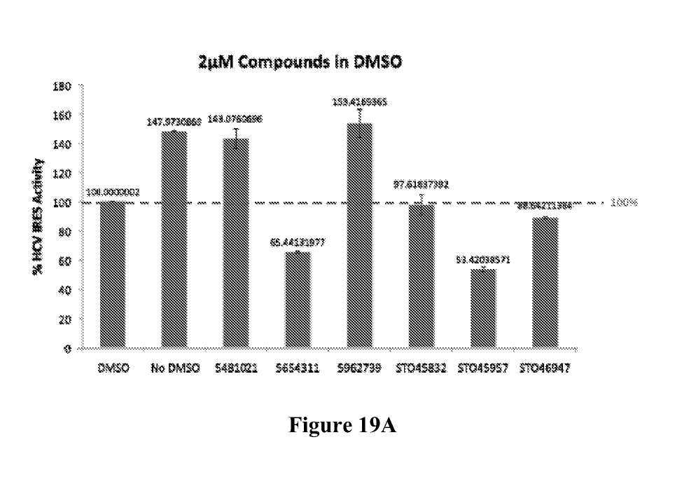

From the 960 small molecules screened from the first 12 trays of a large collection of synthetic organic small molecules, 24 hit compounds were identified. This pilot experiment reveals a 2.5% hit rate. In FIG. 17A, the data reduction is presented in a histogram where % inhibition of HCV IRES is shown as a percentage of control. This data presentation reveals HCV IRES inhibitors (much like the data shown above in HeLa cells when siRNA was used to reduce RPS25 levels). One can see a continuum of mild, significant and marked inhibitor potency within this initial hit series. A subset of the small molecules shared a common structure or scaffold in this initial hit series (FIG. 18).

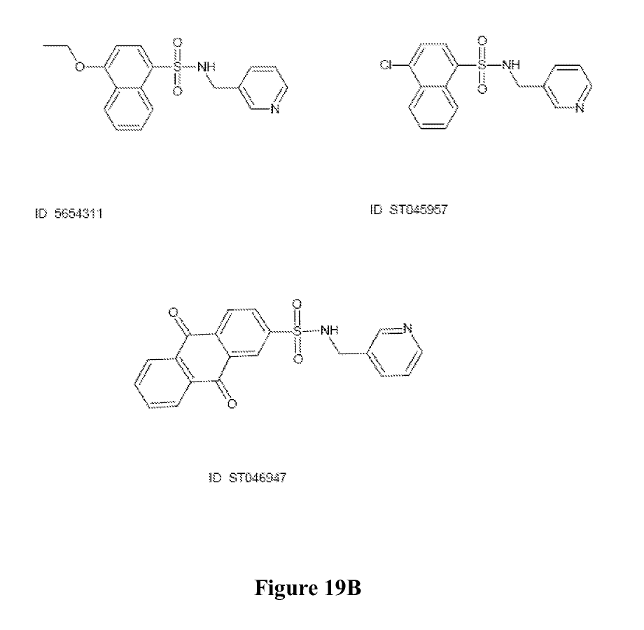

Of the identified inhibitors, novel compounds were found to both inhibit HCV IRES-mediated translation in a concentration dependent manner (FIG. 17B) and inhibit HCV replication in Huh7 cells at 2 μM concentration (FIG. 17C). The compounds all shared a similar structure and were validated in an independent assay from the high throughput screen (FIG. 17D). An additional screen carried out as described above led to the identification of three more compounds that inhibit IRES mediated translation (FIGS. 19A). The structure of the compounds are shown in FIG. 19B.

1. A compound of the following formula: 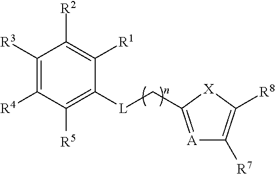
or a pharmaceutically acceptable salt of prodrug thereof, wherein:
A is CR⁹ or N;
L is —O—CR¹⁰R¹¹C(O)—NR⁶—, —NR¹²—NR⁶—, —C(O)—NR⁶—, —SO₂—NR⁶—, —CH₂—NR⁶—, —CH₂—CH₂—NR⁶—, or a substituted or unsubstituted heteroaryl;
n is 0, 1, or 2;
X is —CR¹³═CR¹⁴—, —N═CR¹⁵—, —CR¹⁵═N—, NR¹⁶, O, or S;
R¹, R², R³, R⁴, R⁵, R⁷, R⁸, R⁹, R¹⁰, R¹¹, R¹³, R¹⁴, and R¹⁵ are each independently selected from hydrogen, halogen, hydroxyl, trifluoromethyl, substituted or unsubstituted thio, substituted or unsubstituted alkoxyl, substituted or unsubstituted aryloxyl, substituted or unsubstituted amino, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl; and
R⁶, R¹², and R¹⁶ are each independently selected from hydrogen, substituted or unsubstituted C₁₋₁₂ alkyl, substituted or unsubstituted C₂₋₁₂ alkenyl, substituted or unsubstituted C₂₋₁₂ alkynyl, substituted or unsubstituted C₁₋₁₂ heteroalkyl, substituted or unsubstituted C₂₋₁₂ heteroalkenyl, substituted or unsubstituted C₂₋₁₂ heteroalkynyl, substituted or unsubstituted cycloalkyl, substituted or unsubstituted heterocycloalkyl, substituted or unsubstituted aryl, or substituted or unsubstituted heteroaryl, or substituted or unsubstituted carbonyl.

2. The compound of claim 1, wherein R¹ and R², R² and R³, R³ and R⁴, or R⁵ and R⁶ are combined to form a substituted or unsubstituted aryl, substituted or unsubstituted heteroaryl, substituted or unsubstituted cycloalkyl, substituted or unsubstituted cycloalkenyl, substituted or unsubstituted cycloalkynyl, substituted or unsubstituted heterocycloalkyl, substituted or unsubstituted heterocycloalkenyl, or substituted or unsubstituted heterocycloalkynyl.

3-5. (canceled)

6. The compound of claim 1, wherein A is CH or N.

7. The compound of claim 1, wherein L is a substituted or unsubstituted pyrazole.

8. The compound of claim 1, wherein R³ is ethoxy, dimethylamino, or chloro.

9. The compound of claim 1, wherein X is S or —CH═CH—.

10. The compound of claim 1, wherein the compound is selected from the group consisting of

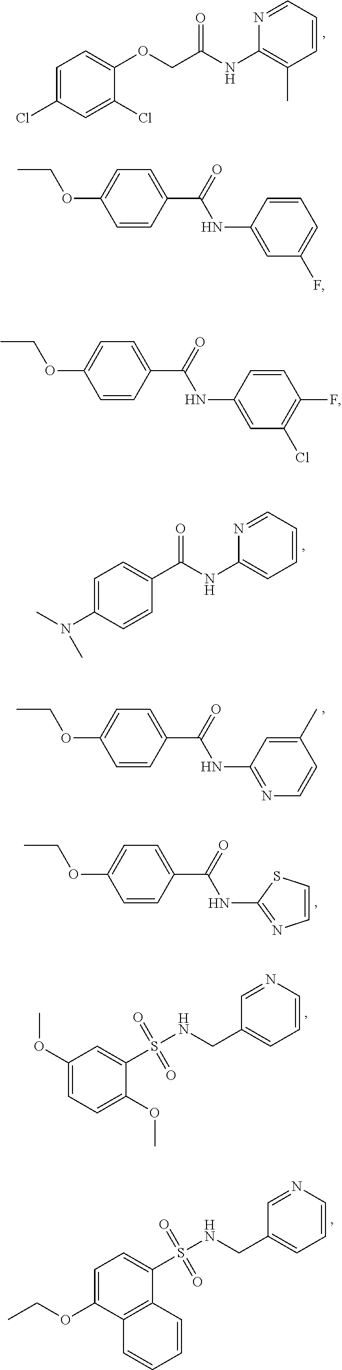

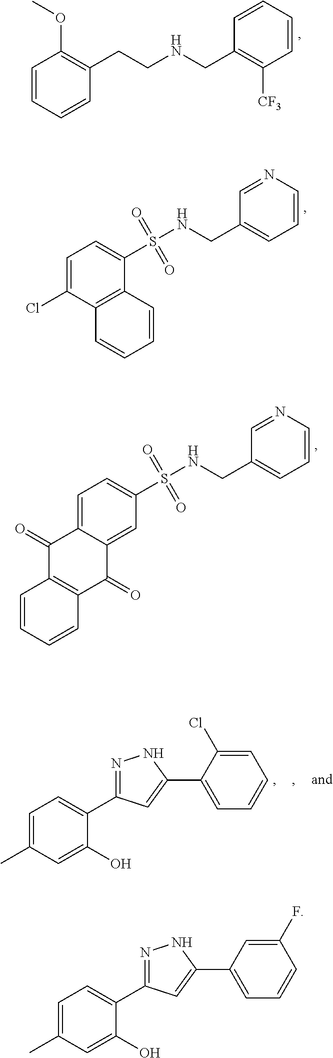

11-22. (canceled)

23. A method of treating or preventing a viral infection in a subject, the method comprising:
(a) identifying a subject with or at risk of developing a viral infection, wherein the viral infection is mediated by a virus comprising an IRES-containing RNA molecule;
(b) administering to the subject a therapeutically effective amount of the compound of claim 1.

24. The method of claim 23, wherein the compound reduces ribosomal protein S25 (Rps25) expression or function in the subject in comparison to a control.

25. The method of claim 23, further comprising administering to the subject a therapeutically effective amount of an agent that reduces ribosomal protein S25 (Rps25) expression or function in the subject in comparison to a control.

26-27. (canceled)

28. The method of claim 25, wherein the agent is a nucleic acid molecule selected from the group consisting of an antisense molecule, a short-interfering RNA (siRNA) molecule, a microRNA (miRNA) molecule, a RNA aptamer, or a combination thereof.

29. (canceled)

30. The method of claim 28, wherein the siRNA molecule comprises SEQ ID NO:5.

31. The method of claim 23, wherein the virus is selected from the group consisting of the a virus within the Picornaviridae Family, a virus within the Dicistroviridae Family, a virus within the Flaviviridae Family, a virus within the Herpesviridae Family, a virus within the Retroviridae Family, and a virus within the Poxviridae Family.

32. (canceled)

33. The method of claim 31, wherein the virus comprises a virus within the Dicistroviridae Family and is selected from the group consisting of a cricket paralysis virus, a taura syndrome virus, and an Israel acute paralysis virus.

34. (canceled)

35. The method of claim 31, wherein the virus comprises a virus within the Flaviviridae Family and is hepatitis C virus (HCV).

36. A method of treating or preventing a viral infection in a subject, the method comprising:
(a) identifying a subject with or at risk of developing a viral infection, wherein the viral infection is mediated by a virus comprising an IRES-containing RNA molecule; and
(b) administering to the subject an effective amount of a therapeutic agent, wherein the agent reduces ribosomal protein S25 (Rps25) expression or function in the subject in comparison to a control.

37-46. (canceled)

47. A method of inhibiting internal ribosome entry site (IRES)-mediated translation, the method comprising:
(a) providing a cell, wherein the cell comprises an IRES-containing RNA molecule; and
(b) contacting the cell with an agent that reduces ribosomal protein S25 (Rps25) expression or function, reduction of Rps25 expression or function as compared to a control indicates the agent inhibits IRES mediated translation.

48. The method of claim 47, further comprising determining that IRES-mediated translation is inhibited by detecting a reduced level of protein expressed by the IRES-containing RNA molecule in comparison to a control.

49-58. (canceled)

59. A method of treating or preventing cancer in a subject, the method comprising:
(a) identifying a subject with or at risk for developing cancer, wherein the cancer is related to increased internal ribosome entry site (IRES)-mediated translation of a mRNA molecule; and
(b) administering to the subject a therapeutically effective amount of the compound of claim 1.

60-66. (canceled)

67. A method of treating or preventing cancer in a subject, the method comprising:
(a) identifying a subject with or at risk of developing cancer, wherein the cancer is related to increased or decreased internal ribosome entry site (IRES)-mediated translation of a cellular mRNA; and
(b) administering to the subject an effective amount of a therapeutic agent, wherein the agent increases or reduces ribosomal protein S25 (Rps25) expression or function in the subject in comparison to a control.

68-76. (canceled)

77. A method of screening for an agent that inhibits or promotes internal ribosome entry site (IRES)-mediated translation, the method comprising:
(a) providing a system comprising a ribosomal protein S25 (Rps25) or a nucleic acid that encodes Rps25 and an IRES-containing RNA molecule;
(b) contacting the system with the agent to be screened; and
(c) determining Rps25 expression or function, wherein a decrease in the level of Rps25 expression or function indicates the agent inhibits IRES-mediated translation, and wherein an increase in the level of Rps25 expression or function indicates the agent promotes IRES-mediated translation.

78-80. (canceled)

81. An agent isolated by the method of claim 77.

82. A method of identifying IRES-containing cellular RNA molecules, the method comprising:
(a) inhibiting Rps25 expression or function in a cell;
(b) determining a protein expression pattern in the cell; and
(c) comparing the protein expression pattern in the cell to a control, wherein a decrease in protein expression of a cellular RNA molecule as compared to a control indicates the cellular RNA molecule contains an IRES.

83. A method of promoting internal ribosome entry site (IRES)-mediated translation, the method comprising:
(a) providing a cell, wherein the cell comprises an IRES-containing RNA molecule; and
(b) contacting the cell with an agent that increases ribosomal protein S25 (Rps25) expression or function, wherein an increase in Rps25 expression or function as compared to a control indicates that the agent promotes IRES-mediated translation.

84-85. (canceled)

86. A method of detecting cancer in a subject, the method comprising:
(a) determining the levels of Rps25 expression in a subject;
(b) comparing the levels of Rps25 to a standard; and
(c) determining the presence of cancer.
